(19) United States
(12) Patent Application Publication
(10) Pub. No.: US 2005/0266544 A1
(43) Pub. Date: Dec. 1, 2005

(54) Methods and compositions for increasing viral vector production in packaging cell lines

(75) Inventors: Won-Bin Young, Des Moines, IA (US); Charles J. Link JR., Clive, IA (US); Tatiana Seregina, West Des Moines, IA (US)

Correspondence Address: MCKEE, VOORHEES & SEASE, P.L.C., 801 GRAND AVENUE, SUITE 3200, DES MOINES, IA, 50309-2721, US

(73) Assignee: Human Gene Therapy Research Institute, Des Moines, IA (US)

(21) Appl. No.: 11/037,398

(22) Filed: Jan. 18, 2005

Related U.S. Application Data
(63) Continuation of application No. 09/560,803, filed on Apr. 28, 2000.
(60) Provisional application No. 60/131,745, filed on Apr. 30, 1999.

(51) Int. Cl. C12P 21/06; C12N 7/00; C12N 7/01; C12N 15/00; C12N 15/09; C12N 15/63; C12N 15/70; C12N 15/74

(52) U.S. Cl. 435/235.1; 435/320.1; 435/69.1

(57) ABSTRACT

Methods and compositions are disclosed to increase viral titer of vector producing cell lines and to reduce potential for re-infection by inhibiting deactivation of helper virus. According to the invention, methylation of helper virus and concomitant helper virus inactivation is directly correlated with increased super-infection and decreased vector production. Novel helper virus, packaging and producing cells and viral vectors are disclosed with improved safety and stability by decreasing helper virus inactivation.

### CROSS-REFERENCE TO RELATED APPLICATIONS

This application claims priority under 35 U.S.C. § 119 of a provisional application Ser. No. 60/131,745 filed Apr. 30, 1999, and from U.S. application Ser. No. 09/560,803 filed Apr. 28, 2000, which applications are hereby incorporated by reference in their entirety.

### FIELD OF THE INVENTION

This invention relates generally to genetic engineering and more specifically to improvements in components and methods used in genetic engineering, namely viral vector production. Vectors produced by the teachings herein can be used in any of a number of molecular protocols including in vitro, ex vivo or in vivo modification of nucleotide sequences present in cells.

### BACKGROUND OF THE INVENTION

Retrovirus is classified by the reverse transcription of its genomic RNA transcript during replication cycle. After infection of the target cell, retroviruses convert their single stranded RNA genome to double stranded DNA (proviral DNA) by the activity of viral reverse transcriptase (RT). The proviral DNA then is inserted into the cellular genome as a provirus.

Integrated provirus in the host chromosome is as stable as host genome sequences and can replicate with the host chromosomes during the cell proliferation. Even more, once the provirus integrated into the germline cells and then can be transmitted from one generation to the next. There is no any specific excision mechanism to delete a provirus from the host genome, but occasionally, a provirus can be deleted with the junction chromosomal DNA or, more commonly, the recombination between LTRs. These events are very rare and have been estimated as low as 4-5×10⁻⁶ events per generation in DBA mice, and about 10−7 in tissue culture cells.

The stable integration of provirus has become an attractive feature to the gene therapy of genetic diseases. Currently, retroviral vectors are the most commonly used gene delivery vehicles in human gene therapy trials. Permanent modifications of a targeted cell's genotype by the integration of a retroviral vector genetic information and the consistent production of retroviral vectors from permanent packaging cells without helper virus are the most significant advantages of retroviral vector systems over other viral vectors. These stable virus producer cells can produce high titer of retroviral vectors in the in vitro cell culture condition.

For long periods of culture, however the stability of vector producer cell genotype becomes a questionable issue, since the possible re-infection of vector on vector producer cells (VPC) themselves and random integrations of vectors can increase risk of mutagenesis. This is particularly so in view of some gene therapy clinical trials, where VPC have been planned to be implanted into tumor lesions in the brain and intraperitoneal cavity of human subjects, therefore, the stability of VPC genotypes is a biosafety concern of gene therapy.

Another issue is the potential recombination of helper virus from a VPC with a vector to produce a replication competent vector (RCR). The formation of RCR has been shown to occur by abnormal template switches between helper virus and retroviral vector sequences during reverse transcription (Otto E et al. Characterization of a replication-competent retrovirus resulting from recombination of packaging and vector sequences. Hum Gene Ther 1994; 5: 567-75; Vanin E F, Kaloss M, Broscius C, Nienhuis A W. Characterization of replication-competent retroviruses from nonhuman primates with virus-induced T-cell lymphomas and observations regarding the mechanism of oncogenesis. J Virol 1994; 68: 4241-50) Therefore, the prevention of RCR outbreak from vector producer cells (VPC) may require attempts to block or decrease RT enzyme activities inside of VPC.

There are two categories of RT enzyme activity inside of VPC. 1) Exogenous viral RT is imported into cells by virus infection. In a cultured VPC, the only source of exogenous RT enzyme activity would be derived from the re-entry of virions produced by the VPC (superinfection of vector). 2) Endogenous RT is a combination of retrovirus-encoded Pr180^{gag-pol} or pol gene product of virion particles within the cell (Katoh I et al. Murine leukemia virus mutations: protease region required for conversion from “immature” to “mature” core form and for virus infectivity. Virology 1985; 63: 280-292; Crawford S, Goff S P. A deletion mutation in the 5′ part of the pol gene of Moloney murine leukemia virus blocks proteolytic processing of the gag and pol poly-proteins. J. Virol. 1985; 53: 899-907; Peng C, Ho B K, Chang T W, Chang N T. Role of human immunodeficiency virus type 1-specific protease in core protein maturation and viral infectivity. J Virol. 1989; 63: 2550-6; Tchenio T, Heidmann T. Defective retroviruses can disperse in the human genome by intracellular transposition. J Virol 1991; 65: 2113-8), endogenous retroviruses (Goodchild N L, Freeman J D, Mager D L. Spliced HERV-H endogenous retroviral sequences in human genomic DNA: evidence for amplification via retrotransposition. Virology 1995; 206: 164-73; Lefebvre S et al. Isolation from human brain of six previously unreported cDNAs related to the reverse transcriptase of human endogenous retroviruses. AIDS Res Hum Retroviruses 1995; 11: 231-7) and retrotransposons (Jensen S, Heidmann T. An indicator gene for detection of germline retrotransposition in transgenic Drosophila demonstrates RNA-mediated transposition of the LINE I element. EMBO J 1991; 10: 1927-37; Jensen S, Gassama M P, Heidmann T. Retrotransposition of the Drosophila LINE I element can induce deletion in the target DNA: a simple model also accounting for the variability of the normally observed target site duplications. Biochem Biophys Res Commun 1994; 202: 111-9; Heidmann O, Heidmann T. Retrotransposition of a mouse IAP sequence tagged with an indicator gene. Cell 1991; 64: 159-70). These different sources of endogenous RT enzyme activity can result in intracellular retrotransposition activity (Lower R, Lower J, Kurth R. The viruses in all of us: characteristics and biological significance of human endogenous retrovirus sequences. Proc Natl Acad Sci USA 1996; 93: 5177-84).

Super infection, and concomitant exogenous RT activity can be limited by interference between viral Env proteins and viral receptors on the cell surface (Env-receptor interference) (Odawara T et al. Threshold number of provirus copies required per cell for efficient virus production and interference in Moloney murine leukemia virus-infected NIH 3T3 cells. J Virol 1998; 72: 5414-24). The re-infection ratio of amphotropic Moloney murine leukemia virus (Am-MoMLV) vector virions on target cells, which were previously infected with wild type Am-MoMLV, is as low as 10⁻⁴ events per cell on rat 208 cells and 10⁻⁶ events per cell on NIH3T3 cells (Miller A D, Chen F. Retrovirus packaging cells based on 10A1 murine leukemia virus for production of vectors that use multiple receptors for cell entry. J Virol. 1996; 70: 5564-71). In contrast to Am-MoMLV re-infection, intracellular retrotransposition of an Env-defective C-type MoMLV has been reported at a higher frequency of 2.7×10⁻⁴ events per cell on feline G355-5 cells (Tchenio T, Heidmann T. High-frequency intracellular transposition of a defective mammalian provirus detected by an in situ calorimetric assay. J Virol 1992; 66: 1571-8).

Retroviral vectors also have high mutation rates and are subject to recombination events that lead to recombinant virus and gene inactivation. In a single retroviral replication cycle, a therapeutic herpes simplex virus (HSV) thymidine kinase (tk) gene was inactivated in approximately 8% of Moloney murine leukemia virus (MoMLV)-based vectors. In the same vector system, the mutation rates within a single retroviral vector were calculated as high as 3% per kb (Parthasarathi, S., A. Varela-Echavarria, Y. Ron, B. D. Preston, and J. P. Dougherty. 1995. Genetic rearrangements occurring during a single cycle of murine leukemia virus vector replication: characterization and implications. J. Virol. 69:7991-8000; Varela-Echavarria, A., C. M. Prorock, Y. Ron, and J. P. Dougherty. 1993. High rate of genetic rearrangement during replication of a Moloney murine leukemia virus-based vector. J Virol. 67:6357-64). In addition to the deletion of HSVtk from retroviral vector, deletion mutations have been observed in retroviral vectors carrying various genes including nerve growth factor receptor (Mavilio, F., G. Ferrari, S. Rossini, N. Nobili, C. Bonini, G. Casorati, C. Traversari, and C. Bordignon. 1994. Peripheral blood lymphocytes as target cells of retroviral vector-mediated gene transfer. Blood. 83:1988-97), α-rev sequence (Junker, U., E. Bohnlein, and G. Veres. 1995. Genetic instability of a MoMLV-based antisense double-copy retroviral vector designed for HIV-1 gene therapy. Gene Ther. 2:639-46), human glucocerebrosidase (Weinthal, J., J. A. Nolta, X. J. Yu, J. Lilley, L. Uribe, and D. B. Kohn. 1991. Expression of human glucocerebrosidase following retroviral vector-mediated transduction of murine hematopoietic stem cells. Bone Marrow Transplant. 8:403-12) and luciferase (Schott, B., E. S. Iraj, and I. B. Roninson. 1996. Effects of infection rate and selection pressure on gene expression from an internal promoter of a double gene retroviral vector. Somat Cell Mol Genet. 22:291-309). It is often difficult to distinguish mutant vector from correct vector sequences in the genomic DNA of vector producer cells (VPC) since both vectors share large homologous DNA sequences except at the deletion or mutation site. To define the mutated region requires multiple Southern blots and analysis by restriction endonuclease mapping. This analysis can be complicated by interference with endogenous retroviral element sequences in the mammalian genome since these sequences are highly homologous to vector sequences.

As can be seen from the foregoing a need exists in the art for methods of increasing the stability of retroviral vectors, and particularly vector producing cells for production of recombinant vectors, thus increasing the safety and usefulness of the same by decreasing viral inactivation, decreasing the potential for super-infection and replication competent virus, and, ideally increasing viral titer of VPC.

It is an object of the present invention to provide methods and compositions for reducing the rate of vector super-infection by maintaining env-receptor interference in vector producer cells.

It is another object of the present invention to provide for vector packaging with improved efficiency by reducing the presence of inactivated helper virus.

It is another object of the present invention to promote vector packaging cell stability by preventing increased vector copy numbers associated with re-infection and vector recombination.

It is yet another object of the invention to provide for high titer production of vector particles to rates as high as 1.5×10⁷ cfu/ml or greater.

Other objects of the invention will become apparent from the detailed description of the invention which follows.

### SUMMARY OF THE INVENTION

The present invention involves methods and strategies for improving vector production efficiency of vector packaging cells and other helper virus mediated vector production protocols. According to the invention, it has been discovered that DNA methylation of helper virus sequences is correlated with inactivation of helper virus gene expression in vector packaging cells. This leads to a cascade of events causing multiple vector integration and decreased or complete loss of vector production.

According to the invention, methods are employed to decrease the presence of or inhibit the effects of inactivated helper virus in vector producer cells. Applicants have discovered that the long terminal repeat promoter sequence traditionally used in helper virus gets preferentially methylated resulting in inactive helper virus. Without active helper virus, viral assembly of recombinant vectors is decreased but also super-infection is increased by reducing the env receptor interference necessary to inhibit viral vector re-entry. This can lead to recombination, and the potential for replication competent virus as well as mutation and gene inactivation of vectors. Any helper virus protocol using a helper virus with a long terminal repeat promoter that has a proclivity to become methylated according to characteristics described herein and known to those of skill in the art can be used in accordance with the teachings herein to improve vector production efficiency and to inhibit reinfection.

Any number of ways of restricting methylation or of reducing the presence of methylated helper virus are also intended to be included herein, including but not limited to: treatment of vector producer cells with 5-aza-C, insertion of a demethylation fragment of murine Thy-1 in front of the 5′ long terminal repeat, ligation of an internal ribosome entry site with a selection marker so that drug selection would ensure promoter function, use of immune response selection, design of synthetic viral promoters to omit methylation sites, screening for other drugs which inhibit methylation, and even antisense inhibition of the human methylase gene which is known and readily accessible through sources such as GenBank.

In one embodiment helper virus plasmids are constructed which enable the positive selection of only cells with active helper virus. More particularly an internal ribosome entry site along with a marker selection gene downstream of the gag, pol, and env genes provide for positive selection of helper virus which has not been inactivated by methylation. General transformation techniques including construction and use of vectors, helper virus, and vector packaging cell lines are all known to those of skill in the art and are also described in the references disclosed and incorporated herein.

The following is a list of definitions useful for describing the invention and helpful in understanding the general techniques of vector production.

As used herein the term “helper virus” shall include any packaging deficient vector or nucleotide sequence encoding a viral protein, the expression of which is necessary in a vector producing cell for assembly and packaging of a particular viral vector capsid. The term helper virus as used herein also includes at least one viral protein operably linked to a viral promoter region capable of being methylated and inactivated, as identified by the teachings and assays disclosed herein.

As used herein the term “retrovirus” is intended to include all virus capable of serving as a vector. This includes but is not limited to retroviral vectors, adenoviral vectors, adeno-associated viral vectors, lentivirus vectors (human and other including porcine), Herpes virus vectors, Epstein-Barr virus vectors, SV40 virus vectors, pox virus vectors, pseudotype virus vectors, which require the use of a helper virus or vector packaging cell line to form infectious viral particles, and which helper virus or packaging cell line may become inactivated due to methylation of promoter sequences associated with the genes necessary for viral assembly. Examples of retroviral vectors include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

The term “viral vector” shall include any viral based vector which embodies less than all structural proteins necessary for viral capsid assembly, and any additional nucleotide sequences desirable for expression or to be delivered to a host cell. The viral vector typically includes foreign DNA which is desired to be inserted in a host cell and usually includes an expression cassette. The foreign DNA can comprise an entire transcription unit, promoter-gene-poly A or the vector can be engineered to contain promoter/transcription termination sequences such that only the gene of interest need be inserted. These types of control sequences are known in the art and include promoters for transcription initiation, optionally with an operator along with ribosome binding site sequences. Examples of such systems include beta-lactase (penicillinase) and lactose promoter systems, (Chang et al., Nature, 1977, 198:1056); the Tryptophan (trp) promoter system (Goeddel, et al., Nucleic Acid Res., 1980, 8:4057) and the lambda derived Pl promoter and N-gene ribosome binding site (Shimatake et al., Nature 1981, 292:128). Other promoters such as cytomegalovirus promoter or Rous Sarcoma Virus can be used in combination with various ribosome elements such as SV40 poly A. The promoter can be any promoter known in the art including constitutive, (supra) inducible, (tetracycline-controlled transactivator (tTA)-responsive promoter (tet system, Paulus, W. et al., “Self-Contained, Tetracycline-Regulated Retroviral Vector System for Gene Delivery to Mammalian Cells”, J of Virology, January 1996, Vol. 70, No. 1, pp. 62-67)), or tissue specific, (such as those cited in Costa, et. Al., European journal of Biochemistry, 258 “Transcriptional Regulation Of The Tissue-Type Plasminogen Activator Gene In Human Endothelial Cells: Identification Of Nuclear Factors That Recognize Functional Elements In The Tissue-Type Plasminogen Activator Gene Promoter” pgs, 123-131 (1998); Fleischmann, M., et. al., FEBS Letters 440 “Cardiac Specific Expression Of The Green Fluorescent Protein During Early Murine Embryonic Development” pgs. 370-376, (1998); Fassati, Ariberto, et. Al., Human Gene Therapy, (9:2459-2468) “Insertion Of Two Independent Enhancers In The Long Terminal Repeat Of A Self Inactivating Vector Results In High-Titer Retroviral Vectors With Tissue Specific Expression” (1998); Valerie, Jerome, et. Al. Human Gene Therapy 9:2653-2659, “Tissue Specific Cell Cycle Regulated Chimeric Transcription Factors For The Targeting Of Gene Expression To Tumor Cells, (1998); Takehito, Igarashi, et. Al., Human Gene Therapy 9:2691-2698, “A Novel Strategy Of Cell Targeting Based On Tissue-Specific Expression Of The Ecotropic Retrovirus Receptor Gene”, 1998; Lidberg, Ulf et.al. The Journal of Biological Chemistry 273, No. 47, “Transcriptional Regulation Of The Human Carboxyl Ester Lipase Gene In Exocrine Pancreas” 1998; Yu, Geng-Sheng et. Al., The Journal of Biological Chemistry 273 No. 49, “Co-Regulation Of Tissue-Specific Alternative Human Carnitine Palmitoyltransferase IB Gene Promoters By Fatty Acid Enzyme Substrate” (1998)). These types of sequences are well known in the art and are commercially available through several sources, ATCC, Pharmacia, Invitrogen, Stratagene, Promega.

The vector is constructed such that the majority of sequences coding for the structural genes of the virus are deleted and replaced by the foreign sequence(s) of interest. The viral structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

The foreign sequence may be incorporated into the proviral backbone in several general ways. The most straightforward constructions are ones in which the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Efforts have been directed at minimizing the viral component of the viral backbone, largely in an effort to reduce the chance for recombination between the vector and the packaging-defective helper virus within packaging cells. A packaging-defective helper virus is necessary to provide the structural genes of a retrovirus, which have been deleted from the vector itself.

In one embodiment, the retroviral vector may be one of a series of vectors described in Bender, et al., J. Virol. 61:1639-1649 (1987), based on the N2 vector (Armentano, et al., J. Virol., 61:1647-1650) containing a series of deletions and substitutions to reduce to an absolute minimum the homology between the vector and packaging systems. These changes have also reduced the likelihood that viral proteins would be expressed. In the first of these vectors, LNL-XHC, there was altered, by site-directed mutagenesis, the natural ATG start codon of gag to TAG, thereby eliminating unintended protein synthesis from that point.

In Moloney murine leukemia virus (MoMuLV), 5′ to the authentic gag start, an open reading frame exists which permits expression of another glycosylated protein (pPr80^{gag}). Moloney murine sarcoma virus (MoMuSV) has alterations in this 5′ region, including a frameshift and loss of glycosylation sites, which obviate potential expression of the amino terminus of pPr80^{gag}. Therefore, the vector LNL6 was made, which incorporated both the altered ATG of LNL-XHC and the 5′ portion of MoMuSV. The 5′ structure of the LN vector series thus eliminates the possibility of expression of retroviral reading frames, with the subsequent production of viral antigens in genetically transduced target cells. In a final alteration to reduce overlap with packaging-defective helper virus, Miller has eliminated extra env sequences immediately preceding the 3′ LTR in the LN vector (Miller, et al., Biotechniques, 7:980-990, 1989).

The paramount need that must be satisfied by any gene transfer system for its application to gene therapy is safety. Safety is derived from the combination of vector genome structure together with the packaging system that is utilized for production of the infectious vector. Miller, et al. have developed the combination of the pPAM3 plasmid (the packaging-defective helper genome) for expression of retroviral structural proteins together with the LN vector series to make a vector packaging system where the generation of recombinant wild-type retrovirus is reduced to a minimum through the elimination of nearly all sites of recombination between the vector genome and the packaging-defective helper genome (i.e. LN with pPAM3).

In one embodiment, the retroviral vector may be a Moloney Murine Leukemia Virus of the LN series of vectors, such as those hereinabove mentioned, and described further in Bender, et al. (1987) and Miller, et al. (1989). Such vectors have a portion of the packaging signal derived from a mouse sarcoma virus, and a mutated gag initiation codon. The term “mutated” as used herein means that the gag initiation codon has been deleted or altered such that the gag protein or fragment or truncations thereof, are not expressed.

In another embodiment, the retroviral vector may include at least four cloning, or restriction enzyme recognition sites, wherein at least two of the sites have an average frequency of appearance in eukaryotic genes of less than once in 10,000 base pairs; i.e., the restriction product has an average DNA size of at least 10,000 base pairs. Preferred cloning sites are selected from the group consisting of NotI, SnaBI, SalI, and XhoI. In a preferred embodiment, the retroviral vector includes each of these cloning sites.

A “packaging cell” is a cell that comprises a helper virus. A packaging cell is transduced with a viral vector containing the foreign nucleotide sequence of interest, so that virions may be produced.

A “producer cell” is a cell that comprises a helper virus and a viral vector. The viral vector is employed to transduce a packaging cell to form a producer cell capable of assembly if infectious vector particles. Examples of packaging cells include, but are not limited to the PE501, PA317, Ψ2, Ψ-AM, PA12, T19-14X, VT-19-17-H2, ΨCRE, ΨCRIP, GP+E-86, GP+envAM12, and DAN cell lines. The vector containing the foreign nucleotide sequence may transduce the packaging cells through any means known in the art. Such means include, but are not limited to, electroporation, the use of liposomes, and CaPO₄ precipitation. The producer cells may then be directly administered, whereby the producer cells generate viral particles capable of transducing the recipient cells.

### DESCRIPTION OF THE FIGURES

FIG. 1. Detection of a second mutated vector from LTKOSN.2 vector production. Viral RNA was extracted from virion particle pellet and subjected to Northern blot analysis. (A) Schematic diagram of LTKOSN vector and the probes. LTKOSN contains a HSVtk gene, which was cloned into the EcoRI site of LXSN. Ψ, extended packaging signal region; Neo, neomycin phosphate transferase gene; SV, promoter sequence of simian virus 40 early gene. The probes were produced from LTKOSN vector by the following restriction endonucleases: B, BamHI; Bp, BpmI; E, EcoRI; H, HindIII; K, KpnI; Sa, SacII; Sp, SpeI; St, StuI. (B) Viral supernatant was subjected to 20% sucrose gradient ultracentrifugation (125,000×g) for 2 hr at 4° C. for virion pelleting and then extracted by RNAzol (Biotecx, Houston, Tex.). Viral RNA was subjected to Northern blot analysis on a 1% agarose-0.4M formaldehyde gel. Two populations (4.5-kb and 3.0-kb) of vectors were detected from packaged viral transcripts by packaging signal probe (Ψ) and Neor probe (Neo). Only one population (4.5-kb) of vector was detected by HStk probe (tk) probe.

FIG. 2. Cellular location of episomal DNA in vector producer cells. Nuclear and cytoplasmic fractions were separated from 1×10⁷ of LTKOSN.2 VPC with centrifugation after Triton X-100 detergent treatment to lyse only the cellular membrane but not nuclear membrane (Lindberg, G. L., C. K. Koehler, J. E. Mayfield, A. M. Myers, and D. C. Beitz. 1992. Recovery mitochondrial DNA from blood leukocytes using detergent lysis. Biochem Genet. 30:27-33). Episomal DNA extracted from cytoplasmic fraction was obtained using phenol/chloroform extraction and then ethanol precipitation. Episomal DNA extracted from nuclear fraction was obtained by adding 5M NaCl to nuclei at 4° C. for overnight to precipitate genomic DNA. Supernatant of episomal DNA from nuclear fraction was separated from genomic DNA pellet using centrifugation and then subjected to phenol/chloroform extraction (Hirt, B. 1967. Selective extraction of polyoma DNA from infected mouse cell cultures. J Mol Biol. 26:365-9). The vast majority of episomal DNA was detected in cytoplasmic fraction by Neo probe. Very little episomal DNA was detected in the nuclear fraction. Different salt concentration was used for the nuclear DNA extraction as reflected in the difference in band migration rate.

FIG. 3. Characterization of mutated LTKOSN vector (ΔLTKOSN) by episomal DNA restriction mapping. (A) Episomal DNA was extracted from the cytoplasm fraction of LTKOSN.2 VPC. These episomal DNA samples were treated with ribonuclease A (RNase A, 1 mg/ml, Boehringer Mannheim, Indianapolis, Ind.) to eliminate RNA contamination prior to restriction enzyme digestion. Without restriction digestion (lane 1), digestion with BamHI (lane 2) or BamHI/EcoRI (lane 3), these episomal DNA were transferred onto a nylon membrane and hybridized with different probes (FIG. 1A), LTR, Ψ, tk, SV40 promoter (SV40) and Neo, respectively. Hybridization was performed at 42° C. for 16 hr and membrane was washed in 0.1×SSC at 65° C. for 1 hr. Without restriction endonuclease treatment, LTKOSN vector is 4.5-kb and ΔLTKOSN vector is 3.0-kb in size. The band in lane 3 indicated by asterisk (*) represents signal from integrated proviral DNA sequences in chromosomes detected by Neo probe. Note the absence of tk or SV40 probe hybridization to the ΔLTKOSN vector on lane 1. (B) Schematic map of ΔLTKOSN. Based on the results of episomal DNA Southern analysis, PCR primers were designed to amplify the deletion region for sequence analysis. A 1.5-kb region including the HSVtk gene and the 5′ portion of SV40 promoter was deleted. The EcoRI polylinker is now join with the 3′ portion of SV40 promoter. Drawings are not to scale.

FIG. 4. Construction and cap-independent translation mechanism of chimeric pAM3-IRES-Zeo helper virus. (A) For details see Materials and Methods. Briefly, a 2.8-kb fragment including IRES-Zeo expression cassette, a SV40 polyadenylation signal sequence, bacterial replication origin (ColE1 Ori) and phage replication origin (F1 Ori) was excised from pIRES-Zeo. The ColE1 Ori and ampicillin resistance gene (AmpR) of pPAM3 were replaced with above 2.8-kb IRES-Zeo-containing fragment from pIRES-Zeo. The EM7 prokaryotic promoter located at 5′ end of Zeo gene permits selection for pAM3-IRES-Zeo in bacterial. (B) Genomic RNA of MoMLV contained two internal stop codons at 3′ends of gag and pol genes that terminate cap-dependent translation and allow appropriate ratios of viral structural proteins. In pAM3-IRES-Zeo derived transcripts, ribosomes also recognize IRES sequence and initiate translation from the first AUG codon of Zeo downstream of IRES sequence. A portion of genomic RNA is spliced into env transcripts that are translated in cap-dependent mechanism. SD, splicing donor; SA, splicing acceptor.

FIG. 5. Gene expression of pAM3-IRES-Zeo and vectors in LTKOSN.2 VPC subclones. (A) Northern blot analysis of cellular RNA extracted from pAM3-IRES-Zeo transfected LTKOSN.2 subclones hybridized with env probe. Unspliced MoMLV transcript (gag-pol-env-IRES-Zeo) and spliced RNA (env-IRES-Zeo) were significantly greater than pPAM3 gene expression, which only exhibited spliced env transcripts. (B) Gene expression of LTKOSN and ΔLTKOSN vectors. Hybridization of the same Northern blot membrane with Neo^{r} probe to detect whole-length LTKOSN (4.0 kb) and ΔLTKOSN (2.5 kb) RNA transcripts, and a Neo^{r} transcript (1.2 kb) expressed from the internal SV40 promoter. Fewer vector transcripts were retained in pAM3-IRES-Zeo transfected cells since transcripts were packaged into virions. (C) Hybridization of the same Northern blot membrane with human GAPDH cDNA probe demonstrates fairly equivalent RNA loading.

FIG. 6. DNA methylation of helper virus 5′LTR over time with and without Zeocin™ selection. (A) Schema of pAM3-IRES-Zeo helper virus showing restriction enzyme sites and the probe used for the methylation analysis. B, BstEII; E, EcoRV; S, SmaI; AAA, SV40 polyadenylation signal. Drawing is not to scale. (B) Genomic DNA Southern blot membrane was probed with a 261-bp fragment excised from pAM3-IRES-Zeo with KpnI and AflII digestions. If methylation was present at the SmaI site, a 608-bp fragment would result instead of a 348-bp fragment. The degree of DNA methylation was calculated as the intensity ratio of SmaI insensitive band (608-bp) divided by the sum of the intensity of this 608-bp band and SmaI sensitive fragment (348-bp).

FIG. 7. The effectiveness of Zeocin™ selection on helper virus gene expression in AMIZ cells over time. (A) Unspliced MoMLV transcript (gag-pol-env-IRES-Zeo) and spliced RNA (env-IRES-Zeo) were detected by an env probe in cellular RNA extracted from AMIZ cells with and without continuous Zeocin™ selection on Days 0, 15, 54 and 78. (B) Hybridization with GAPDH cDNA probe demonstrates the relative RNA loading.

FIG. 8. Gene expression of pAM3-IRES-Zeo helper virus and LEIN vector in AMIZ cells transfected with LEIN vector. (A) Northern blot analysis of cellular RNA extracted from AMIZ cells transfected with LEIN vector evaluated at passage 3 (day 0, lane 1) and on days 56 and 67 (lanes 2-5) by hybridization with env probe. Zeocin™ and G418 selection resulted in greater levels of unspliced MoMLV RNA transcript (gag-pol-env-IRES-Zeo) and spliced RNA transcripts (env-IRES-Zeo). (B) The level of LEIN vector was also greater in AMIZ cells under Zeocin™ and G418 selection. (C) Re-hybridization with GAPDH cDNA probe was used to demonstrate fairly equivalent RNA loading.

FIG. 9. Drug selection eliminates DNA methylation of helper virus and vector from VPC population. Genomic DNA extracted from AMIZ cells transfected with LEIN vector with Zeocin™ and G418 or without drug selection on days 0 (lanes 3 and 4), 56 and 67 (lanes 5-12) were first digested with DraI and EcoRV and divided into two equal portions. One portion was subjected to methylation-sensitive SmaI restriction endonuclease digestion and the other without SmaI digestion. (A) Schema of the helper virus and vectors showing the locations of restriction enzyme sites and probes used for methylation analysis. B, BstXI, D, DraI; E, EcoRV; S, SmaI; AAA, SV40 polyadenylation signal. Drawings are not to scale. (B) Hybridization of gag DNA probe to detect helper virus 5′ LTR. SmaI digestion reduced the 1.8-kb band (even numbered lanes 4-12) to 1.5-kb (odd numbered lanes 3-11). DNA from NIH3T3 cells was used to show the presence of endogenous retroviral elements (lanes 1 and 2). Since a 1.8-kb band was generated from endogenous retroviral element after SmaI digestion (lane 1), the values for SmaI resistance were measured by densitometry as the relative intensities of the 1.8-kb bands without SmaI digestion, and 1.5-kb bands after SmaI digestion. (C) GFP DNA probe was used to detect the 5′ LTR of LEIN vector. SmaI digestion reduced the 3.7-kb fragment to 3.4-kb, 2.7-kb and 2.4-kb fragments dependent upon the methylation status of SmaI site in LEIN vector. (D) A 0.68-kb DNA fragment digested from gag gene of pPAM3 by BstXI was used as a probe to detect a 1.2-kb band of endogenous retroviral element to demonstrate relative loading in paired SmaI+/− digestions.

FIG. 10 is a graph showing distribution of VPC microcultures as a function of slot-blot intensity score for VPC derived from A375.AMIZ-1.

### DETAILED DESCRIPTION OF THE INVENTION

Mammalian DNA methyltransferase (MTase) catalyses the transfer of a methyl group to cytosines located 5′ to guanosine (CpG dinucleotide) and causes epigenetic effects which usually involves gene silencing. Methylated CpG dinucleotides inactivate gene expression by altering DNA conformation (Feil, R., M. D. Boyano, N. D. Allen, and G. Kelsey. 1997. Parental chromosome-specific chromatin conformation in the imprinted U2af1-rs1 gene in the mouse. J. Biol. Chem. 272:20893-900; Keshet, I., J. Lieman-Hurwitz, and H. Cedar. 1986. DNA methylation affects the formation of active chromatin. Cell. 44:535-43; Muiznieks, I., and W. Doerfler. 1994. The topology of the promoter of RNA polymerase II- and III-transcribed genes is modified by the methylation of 5′-CG-3′ dinucleotides. Nucleic Acids Res. 22:2568-75) or attracting the binding of methylated CpG-binding proteins (Hendrich, B., and A. Bird. 1998. Identification and characterization of a family of mammalian methyl-CpG binding proteins. Mol. Cell Biol. 18:6538-47; Lamb, B. T., K. Satyamoorthy, L. Li, D. Solter, and C. C. Howe. 1991. CpG methylation of an endogenous retroviral enhancer inhibits transcription factor binding and activity. Gene Expr. 1:185-96; Nan, X., F. J. Campoy, and A. Bird. 1997. MeCP2 is a transcriptional repressor with abundant binding sites in genomic chromatin. Cell. 88:471-81; Nan, X., H. H. Ng, C. A. Johnson, C. D. Laherty, B. M. Turner, R. N. Eisenman, and A. Bird. 1998. Transcriptional repression by the methyl-CpG-binding protein MeCP2 involves a histone deacetylase complex. Nature. 393:386-9) to impede transcription. The majority of DNA methylation patterns in mammalian genomes are found in retrovirus-related sequences, such as retrotransposons and endogenous or exogenous retroviruses (Yoder, J. A., C. P. Walsh, and T. H. Bestor. 1997. Cytosine methylation and the ecology of intragenomic parasites. Trends. Genet. 13:335-40). Evidence suggests that DNA methylation may act as a host defense system against retroviral invasion of the cellular genome (Bestor, T. H., and B. Tycko. 1996. Creation of genomic methylation patterns. Nat. Genet. 12:363-7; Yoder, J. A., and T. H. Bestor. 1996. Genetic analysis of genomic methylation patterns in plants and mammals. Biol. Chem. 377:605-10; Yoder, J. A., supra). DNA methylation can be triggered by insertion of viral DNA sequence into chromosomes regardless of whether DNA transfection (Bednarik, D. P., J. D. Mosca, and N. B. Raj. 1987. Methylation as a modulator of expression of human immunodeficiency virus. J. Virol. 61:1253-7) or viral infection (Jahner, D., and R. Jaenisch. 1985. Retrovirus-induced de novo methylation of flanking host sequences correlates with gene inactivity. Nature. 315:594-7; Mikovits, J. A., H. A. Young, P. Vertino, J. P. Issa, P. M. Pitha, S. Turcoski-Corrales, D. D. Taub, C. L. Petrow, S. B. Baylin, and F. W. Ruscetti. 1998. Infection with human immunodeficiency virus type 1 upregulates DNA methyltransferase, resulting in de novo methylation of the gamma interferon (IFN-gamma) promoter and subsequent downregulation of IFN-gamma production. Mol. Cell Biol. 18:5166-77; Saggioro, D., M. Panozzo, and L. Chieco-Bianchi. 1990. Human T-lymphotropic virus type I transcriptional regulation by methylation. Cancer Res. 50:4968-73) was used to introduce the viral DNA sequences.

In several experimental systems, host cell methylation of retroviral provirus or retrotransposons has been evaluated. In a transgenic mouse model, a retroviral provirus altered the methylation pattern within 1 kb of the retroviral integration site. The provirus was methylated leading to an inactivation of transcription (Jahner, D., and R. Jaenisch. 1985. Chromosomal position and specific demethylation in enhancer sequences of germ line-transmitted retroviral genomes during mouse development. Mol. Cell Biol. 5:2212-20; Jahner, D., H. Stuhlmann, C. L. Stewart, K. Harbers, J. Lohler, I. Simon, and R. Jaenisch. 1982. De novo methylation and expression of retroviral genomes during mouse embryogenesis. Nature. 298:623-8). Sequences of small interspersed repetitive elements contained in the rat α-fetoprotein promoter region were associated with increased DNA methylation and decreased downstream reporter gene expression (Hasse, A., and W. A. Schulz. 1994. Enhancement of reporter gene de novo methylation by DNA fragments from the alpha-fetoprotein control region. J. Biol. Chem. 269:1821-6). Reduction of host DNA methylation leads to amplification and retrotransposition of KERV-1 (kangaroo endogenous retroviral element-1) and xenologous recombination of chromosomes in interspecific mammalian hybrids of the Australian wallaby (O'Neill, R., M. O'Neill, and J. A. Graves. 1998. Undermethylation associated with retroelement activation and chromosome remodelling in an interspecific mammalian hybrid. Nature. 393:68-72). Interestingly, retroviruses may benefit from host DNA methylation as well. HIV-1 infection may induce host DNA methylation activity and, as a consequence, the promoter region of gamma interferon was downregulated by DNA methylation (Mikovits, J. A., supra). This may alter the balance of cytokines and reduce immune surveillance (Mikovits, J. A., Raziuddin, M. Gonda, M. Ruta, N. C. Lohrey, H. F. Kung, and F. W. Ruscetti. 1990. Negative regulation of human immune deficiency virus replication in monocytes. Distinctions between restricted and latent expression in THP-1 cells. J. Exp. Med. 171:1705-20; Mikovits, J. A., supra). The inactivation of HIV-1 or HTLV-1 gene expression by host DNA methylation of viral LTR regions may also induce latency of HIV-1 or HTLV-1 infection (Bednarik, D. P., supra; Saggioro, D., M. Forino, and L. Chieco-Bianchi. 1991. Transcriptional block of HTLV-I LTR by sequence-specific methylation. Virology. 182:68-75; Saggioro, D., supra). These prior experiments did not postulate the drastic events DNA methylation of helper virus 5′LTR in VPC could have on re-infection and viral titer.

Many commonly used retroviral vector packaging cell lines were established by co-transfection of two plasmids, one plasmid contains a helper virus genome and the other encodes a drug selection marker (Markowitz, D., supra; Miller, A. D., supra; Miller, A. D., supra; Miller, A. D., supra). In this co-transfection system, selection for drug resistance does not require active helper virus gene expression, so the 5′LTR promoter region can be silenced by DNA methylation (Young, W.-B., supra). Prior studies have demonstrated the concept of including an antibiotic selection marker (Cosset, F. L., Y. Takeuchi, J. L. Battini, R. A. Weiss, and M. K. Collins. 1995. High-titer packaging cells producing recombinant retroviruses resistant to human serum. J. Virol. 69:7430-6) or a cellular surface FACS marker (human Phoenix cell line, http://www.standford.edu/group/nolan/NL-phoenix.html) at the downstream of gag-pol to monitor the gene expression.

Applicants invention includes techniques and methods which disclose the for the first time the drastic effects helper virus methylation and the ability to identify, select for, and maximize the presence of active helper virus. Any number of ways of restricting methylation or of reducing the presence of methylated helper virus are also intended to be included herein, including but not limited to: treatment of vector producer cells with 5-aza-C, insertion of a demethylation fragment of murine Thy-1 in front of the 5′ long terminal repeat, ligation of an internal ribosome entry site with a selection marker so that drug selection would ensure promoter function, use of immune response selection, design of synthetic viral promoters to omit methylation sites, screening for other drugs which inhibit methylation, and even antisense inhibition of the human methylase gene which is known and readily accessible through sources such as GenBank.

In a most preferred embodiment, a chimeric helper virus, is designed containing a marker selection gene down stream of the helper virus sequences in combination with a picornavirus ribosomal entry site sequence or other similar functioning sequence such that expression of the selection gene only occurs with functional helper virus. Such selectable marker may contain an antibiotic resistance gene, such as those that confer resistance to ampicillin, kanamycin, tetracycline, or streptomycin and the like. These can include genes from prokaryotic or eukaryotic cells such as dihydrofolate reductase or multi-drug resistance I gene, hygromycin B resistance that provide for positive selection. Any type of positive selector marker can be used such as neomycin or Zeosyn and these types of selectors are generally known in the art. Several procedures for insertion and deletion of genes are known to those of skill in the art and are disclosed. For example in Maniantis, “Molecular Cloning”, Cold Spring Harbor Press. See also Post et al., Cell, Vol. 24:555-565 (1981). An entire expression system must be provided for the selectable marker genes and the genes must be flanked on one end or the other with promoter regulatory region and on the other with transcription termination signal (polyadenylation cite). Any known promoter/transcription termination combination can be used with the selectable marker genes. For example SV40 promoter and SV40 poly A.

An internal ribosome entry site (IRES) sequence is present in encephalomyocarditis virus (ECMV) (Jang, S. K., H. G. Krausslich, M. J. Nicklin, G. M. Duke, A. C. Palmenberg, and E. Wimmer. 1988. A segment of the 5′ nontranslated region of encephalomyocarditis virus RNA directs internal entry of ribosomes during in vitro translation. J. Virol. 62:2636-43), a member of picornaviruses (Rueckert, R. R. 1996. Virology. Chapter 21 “Picornaviridae: The viruses and their replication”. 1:609-654).

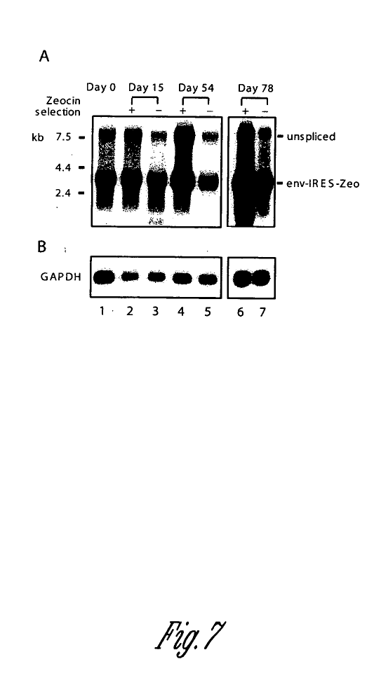

During translation of most eukaryotic mRNAs, ribosomes scan mRNA from the 5′ cap sequence until an initiation codon is reached. In contrast, in picornavirus mRNA, ribosomes initiate translation by an alternative mechanism that involves internal initiation rather than scanning. The IRES sequences of picornavirus enable ribosomes to bind in a cap-independent fashion and start translation at the next AUG codon downstream (Jang, S. K., and E. Wimmer. 1990. Cap-independent translation of encephalomyocarditis virus RNA: structural elements of the internal ribosomal entry site and involvement of a cellular 57-kD RNA-binding protein. Genes Dev. 4:1560-72). Ligation of IRES sequence followed by Zeo at the 3′ end of env gene permits the translation of helper virus open reading frames and a selection marker from this mRNA (FIG. 7). Selection with Zeocin™ eliminates cells with methylated helper virus 5′LTR from the population. This design should ensure sustained helper virus gene expression which would increase virion production and create sufficient Env-receptor interference to prevent superinfection. The prevention of superinfection may in turn reduce RCR formation (Miller, A. D., D. R. Trauber, and C. Buttimore. 1986. Factors involved in production of helper virus-free retrovirus vectors. Somat. Cell Mol. Genet. 12:175-83; Muenchau, D. D., S. M. Freeman, K. Cornetta, J. A. Zwiebel, and W. F. Anderson. 1990. Analysis of retroviral packaging lines for generation of replication-competent virus. Virology. 176:262-5). One additional advantage is that pAM3-IRES-Zeo allows for establishment of packaging cell lines within a shorter time period. This advantage might be critical when making human VPC from a primary cell culture or stem cells to avoid immune rejection (Takeuchi, Y., F. L. Cosset, P. J. Lachmann, H. Okada, R. A. Weiss, and M. K. Collins. 1994. Type C retrovirus inactivation by human complement is determined by both the viral genome and the producer cell. J. Virol. 68:8001-7; Takeuchi, Y., C. D. Porter, K. M. Strahan, A. F. Preece, K. Gustafsson, F. L. Cosset, R. A. Weiss, and M. K. Collins. 1996. Sensitization of cells and retroviruses to human serum by (alpha 1-3) galactosyltransferase. Nature. 379:85-8) while transplantation of VPC into patients is necessary for continuous gene transfer (Ram, Z., K. W. Culver, E. M. Oshiro, J. J. Viola, H. L. DeVroom, E. Otto, Z. Long, Y. Chiang, G. J. McGarrity, L. M. Muul, D. Katz, R. M. Blaese, and E. H. Oldfield. 1997. Therapy of malignant brain tumors by intratumoral implantation of retroviral vector-producing cells. Nat. Med. 3:1354-61).

The viral vector capsids produced by the methods of the invention can be used for any diagnostic or therapeutic genetic engineering protocol including in vitro, ex vivo, or in vivo expression of a desired nucleotide sequence. For example the treatment of cancer such as by the Herpes simplex virus, thymidine kinase gene transfer system Martuza RL et al., “Experimental therapy of human glioma by means of a genetically engineered virus mutant”, Science, 1991; 252:854-856). Also in ex vivo gene therapy protocols such as bone marrow purging (Seth P., et al., “Adenovirus-mediated gene transfer to human breast tumor cells: an approach for cancer gene therapy and bone marrow purging”, Cancer Res. 56(6): 1346-1351 (1996; Andersen, N. S., et al., “Failure of immunologic purging in mantle cell lymphoma assessed by polymerase chain reaction detection of minimal residual disease”, Blood, 90(10):4212-4221 (1997)) thus when the transformed cells are reintroduced to the patient they will generate a decreased immune response. These may also be used for diagnostic purposes as well.

To fully exploit the benefits of the methods and compositions described herein, the use of many general gene therapy improvements are contemplated and are intended to be within the scope of this invention.

All references cited herein are hereby expressly incorporated in their entirety be reference.

### EXAMPLE 1

### Restriction Mapping of Retroviral Vector Episomal DNA

High frequencies of superinfection, (Young, W.-B., G. L. Lindberg, and C. J. Link, Jr. 1999. DNA methylation increased genetic instability of retroviral vector producer cells. In preparation) and retrotransposition, (Young, W.-B., G. L. Lindberg, and C. J. Link, Jr. 1999. High frequency retrotransposition of retroviral vector in cultured vector producer cells. In preparation), of retroviral vectors in cultured VPC results in detectable amounts of episomal DNA. Episomal DNA is advantageous for the Southern analysis of vectors because it is not subject to interference from endogenous retroviral sequences. Episomal vectors or retroviral sequences have been observed with other retroviruses, including mouse mammary tumor virus (Ringold, G. M., K. R. Yamamoto, P. R. Shank, and H. E. Varmus. 1977. Mouse mammary tumor virus DNA in infected rat cells: characterization of unintegrated forms. Cell. 10:19-26), avian sarcoma virus (Varmus, H. E., and P. R. Shank. 1976. Unintegrated viral DNA is synthesized in the cytoplasm of avian sarcoma virus-transformed duck cells by viral DNA polymerase. J Virol. 18:567-73), avian leukosis virus (Robinson, H. L., and B. D. Miles. 1985. Avian leukosis virus-induced osteopetrosis is associated with the persistent synthesis of viral DNA. Virology. 141:130-43), HIV-1 (Pauza, C. D., and J. Galindo. 1989. Persistent human immunodeficiency virus type 1 infection of monoblastoid cells leads to accumulation of self-integrated viral DNA and to production of defective virions. J Virol. 63:3700-7) and in avian packaging cells (Lum, R., and M. L. Linial. 1998. Retrotransposition of nonviral RNAs in an avian packaging cell line. J Virol. 72:4057-64). In this example, applicants successfully identified a HSVtk-deleted vector from VPC by analyzing episomal DNA directly instead of by genomic DNA restriction mapping. PCR primers were therefore designed accordingly to amplify this mutated region. The same primers were used to sequence this deletion without sequencing walking.

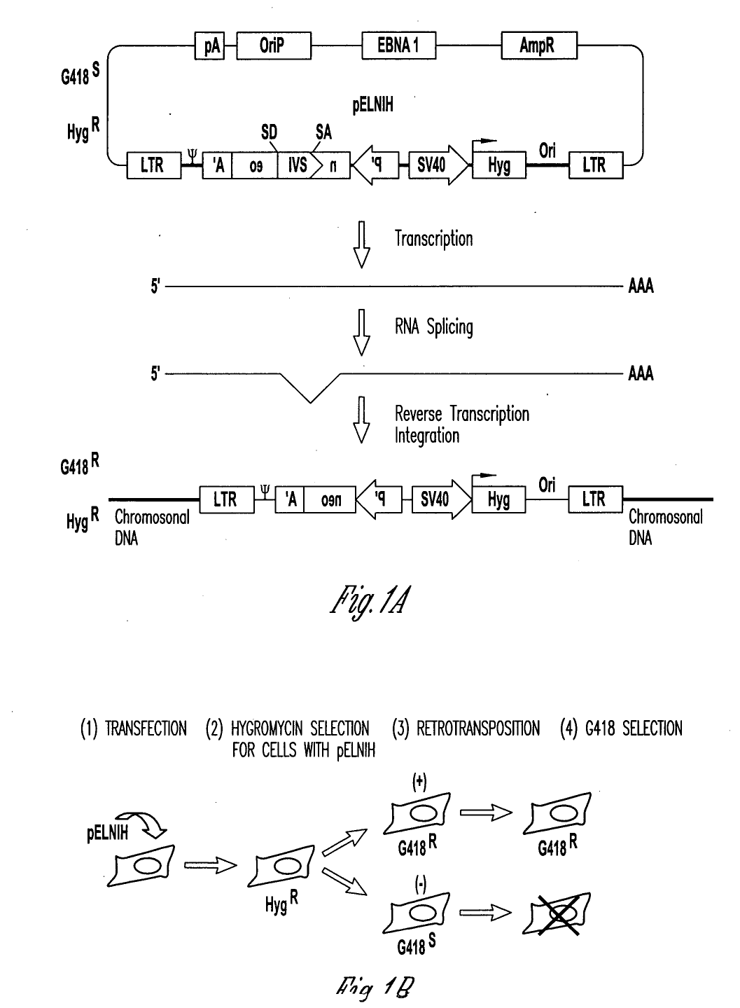

A LTKOSN.2 VPC was previously established in our group for a phase I human gene therapy clinic trial (Link, C. J., Jr., D. Moorman, T. Seregina, J. P. Levy, and K. J. Schabold. 1996. A phase I trial of in vivo gene therapy with the herpes simplex thymidine kinase/ganciclovir system for the treatment of refractory or recurrent ovarian cancer. Hum Gene Ther. 7:1161-79). pLTKOSN plasmid DNA (FIG. 1A) was first introduced into the ecotropic packaging cell line GP+E86 (Markowitz, D., S. Goff, and A. Bank. 1988. A safe packaging line for gene transfer: separating viral genes on two different plasmids. J. Virol. 62:1120-4) by transient transfection. Supernates from these cells were then used to transduce the amphotropic retroviral packaging line PA317 (Miller, A. D., and C. Buttimore. 1986. Redesign of retrovirus packaging cell lines to avoid recombination leading to helper virus production. Mol Cell Biol. 6:2895-902) which were selected in G418 (1 mg/ml) for 2 weeks. Twenty different VPC clones were isolated from the original pool of cells. LTKOSN.2 VPC produces viral titers of approximately 1×10⁶ colony formation unit (cfu)/ml (Link, C. J., Jr., D. Moorman, T. Seregina, J. P. Levy, and K. J. Schabold. 1996. A phase I trial of in vivo gene therapy with the herpes simplex thymidine kinase/ganciclovir system for the treatment of refractory or recurrent ovarian cancer. Hum Gene Ther. 7:1161-79). A deletion of the HSVtk gene in LTKOSN.2 VPC was first detected in viral RNA collected from pelleted viral particles in Northern blot analysis by using different probes (FIG. 1B). This result indicated that the titer calculated from only G418 resistant colonies did not represent the titer of full length LTKOSN vector, which implies that the evaluation of vector titer needs to first insure that all vectors contain an intact HSVtk suicide gene.

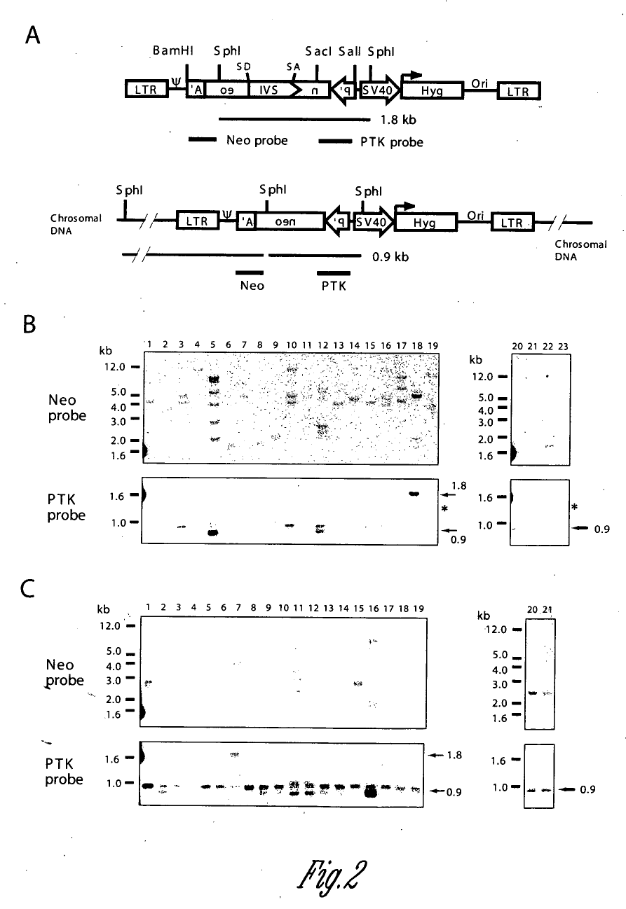

To analyze this mutation without the interference with endogenous retroviral elements present in the cellular genomes, unintegrated, episomal copies of viral DNA were used for Southern blot analysis. Small amounts of episomal DNA derived from vector sequences have been routinely detected within VPC from PA317 and GP+E86 derived VPC. Episomal DNA was extracted from both the cytoplasmic fraction and nuclear fraction of 1×10⁷ LTKOSN.2 VPC. First, cells were trypsinized and subjected to 1% Triton X-100 detergent for 5 min at room temperature to lyse the cellular membrane but not nuclear membrane. Nuclei were separated from the cytoplasmic fraction by centrifugation at 9,500×g for 5 min at 4° C. (Lindberg, G. L., C. K. Koehler, J. E. Mayfield, A. M. Myers, and D. C. Beitz. 1992. Recovery mitochondrial DNA from blood leukocytes using detergent lysis. Biochem Genet. 30:27-33). Cytoplasmic fractions were subjected to phenol/chloroform extraction and ethanol precipitation to isolate purified episomal DNA. The episomal DNA in nuclei was extracted using Hirt's method (Hirt, B. 1967. Selective extraction of polyoma DNA from infected mouse cell cultures. J Mol Biol. 26:365-9) with 5M NaCl to remove genomic DNA. The supernate containing episomal DNA was isolated from cell nuclei by centrifugation (13,000×g for 15 min) and then subjected to phenol/chloroform extraction and ethanol precipitation. Episomal DNA samples extracted from both the cytoplasmic and nuclear fractions were evaluated by Southern analysis. The results clearly show that episomal DNA was mainly detected in the cytoplasmic rather than the nuclear fraction of VPC (FIG. 2).

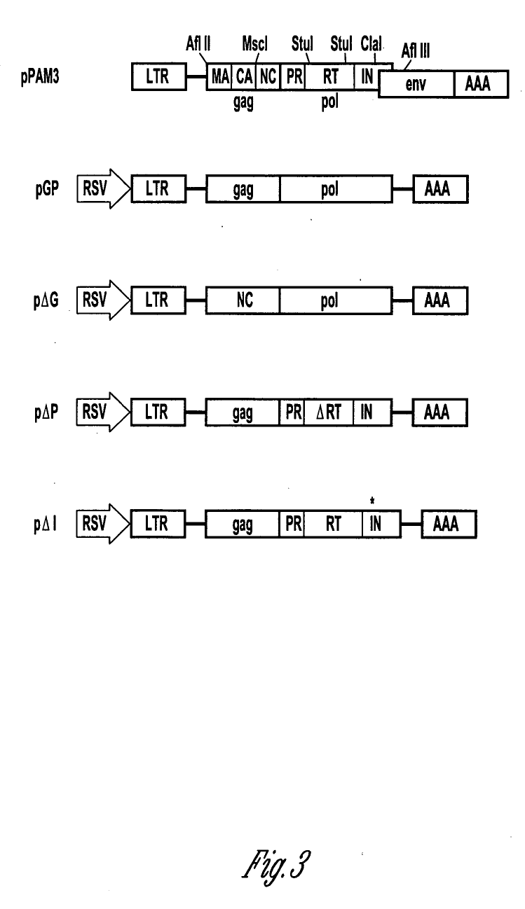

To identify the primary structure of the deleted viral vector, restriction mapping and Southern blot analysis of the retroviral vectors was performed on the episomal DNA extracted from the cytoplasmic fraction of LTKOSN.2 VPC. The same membrane was hybridized at 42° C. with various probes, including long terminal repeat (LTR; SacII/KpnI), extended packaging signal sequence (ψ; SpeI/EcoRI), HSVtk (EcoRI fragment), SV40 promoter (BamHI/StuI), and Neor (HindIII/BpmI), respectively (FIG. 1A). Without restriction enzyme digestion, two respective sizes of episomal vector DNA were detected, 4.5-kb and 3.0-kb. Linear LTKOSN is represented by the 4.5-kb DNA band (FIG. 3A, lane 1). BamHI digestion of the episomal DNA resulted in two fragments, 2.7-kb and 1.8-kb, being generated from the 4.5-kb linear LTKOSN, while the second episomal proviral vector (3.0-kb) was resistant to BamHI digestion (FIG. 3A, lane 2). This suggested that the 3.0-kb DNA (ΔLTKOSN) was a mutant of LTKOSN in which the BamHI site was deleted. The primary structure of this truncated 3.0-kb LTKOSN vector was found to include the 5′ LTR, extended packaging signal (Ψ and portion of gag sequence), the Neo^{r} gene and the 3′ LTR, but did not contain the HSVtk gene and SV40 promoter.

According to these restriction mapping results of retroviral episomal DNA, we designed a pair of primers flanking the suspicious deletion region for PCR amplification. Sequencing was also performed using either one of PCR primers without further sequence walking. Extracted episomal DNA was first PCR amplified using a forward primer (5′-CTG TGT CTG TCC GAT TGT CTA GTG TC-3′) complementary to the extended packaging signal region, and a reverse primer (5′-CCC TTC CCG CTT CAG TGA CAA CG-3′) complementary to the Neo^{r} gene. The PCR included 30 cycles of 1-min denaturation at 94° C., 1-min annealing at 50° C. and 2-min extension at 72° C. The PCR product was then purified by gel electrophoreses and subjected to sequencing analysis. The deletion region in the mutant vector, ΔLTKOSN, included the entire HSVtk gene (except the EcoRI polylinker region at its 5′ end) and most of the 5′ end of SV40 promoter sequence (FIG. 3B). Since only 81 bp of the 3′ end of SV40 promoter remained adjacent to Neor gene, the 0.33-kb SV40 probe, which has only 52 bp overlap with this 81 bp, did not show any detectable signal for ΔLTKOSN vector by episomal DNA Southern analysis (FIG. 3). Therefore, Neo^{r} gene expression was driven by the 5′LTR in ΔLTKOSN vector rather than the remaining, truncated SV40 promoter sequences.

### EXAMPLE 2

### Chimeric Retroviral Helper Virus and Picornavirus IRES Sequence to Eliminate DNA Methylation for Improved Retroviral Packaging Cells

Most retroviral packaging cell lines were established by a helper virus plasmid co-transfected with a separate plasmid encoding a selection marker. Since this selection marker co-existed in trans with the helper virus sequence, helper virus gene expression could be inactivated by host DNA methylation despite selection for the co-transfected selection marker. We have reported that DNA methylation could occur in the LTR region of helper virus in vector producer cells (VPC) up to 2% of the population per day (Young et. al., JVI 1836-99). To overcome host cell DNA methylation that suppresses viral gene expression, we constructed a chimeric retroviral helper virus, pAM3-IRES-Zeo, that contains MoMLV helper virus and a picornavirus internal ribosome entry site (IRES) sequence followed by a Zeocin™ selection marker at the 3′ end of env sequence. This pAM3-IRES-Zeo permitted selection for intact and functional helper virus in transfected cells without subcloning. By selection with Zeocin™, a mixed population of pAM3-IRES-Zeo transfected NIH3T3 cells (AMIZ cells) were maintained with little or no DNA methylation of the helper virus 5′LTR. The high level of pAM3-IRES-Zeo gene expression resulted in no detectable vector superinfection and high vector titers (2×10⁶ to 1.5×10⁷ cfu/ml) after introduction of a retroviral vector. When Zeocin™ selection was withdrawn from AMIZ cells, methylation of the 5′LTR increased from 17% to 36% of the population during 67 days of continuous culture and the cells became susceptible to superinfection. During this period, gene expression of pAM3-IRES-Zeo decreased and vector titer production was reduced to 2×10⁴ cfu/ml. These data demonstrate an important role of DNA methylation in the genetic instability of VPC. The chimeric helper virus allows establishment of a mix-population of packaging cells capable of high level and sustained vector production without cloning procedures.

It has been observed that extensive DNA methylation can occur in murine LTKOSN.2 VPC of retroviral helper virus sequences at 2% of cell population per day. The DNA methylation of helper virus 5′LTR in LTKOSN.2 VPC correlated with reduced helper virus gene expression. These cells had significantly reduced Env-receptor interference and became target cells for vector re-entry (superinfection). The VPC developed increasing genetic instability manifested by increasing vector copy numbers. The decreased helper virus gene expression secondary to DNA methylation dramatically reduced vector titer of VPC (Young, W.-B., G. L. Lindberg, and C. J. Link, Jr. 2000. DNA methylation of helper virus increases genetic instability of retroviral vector producer cells. JVI 1836-99, J. Virol, in press). In order to overcome these limitations caused by host DNA methylation, a retroviral helper virus was designed to improve vector packaging efficiency.

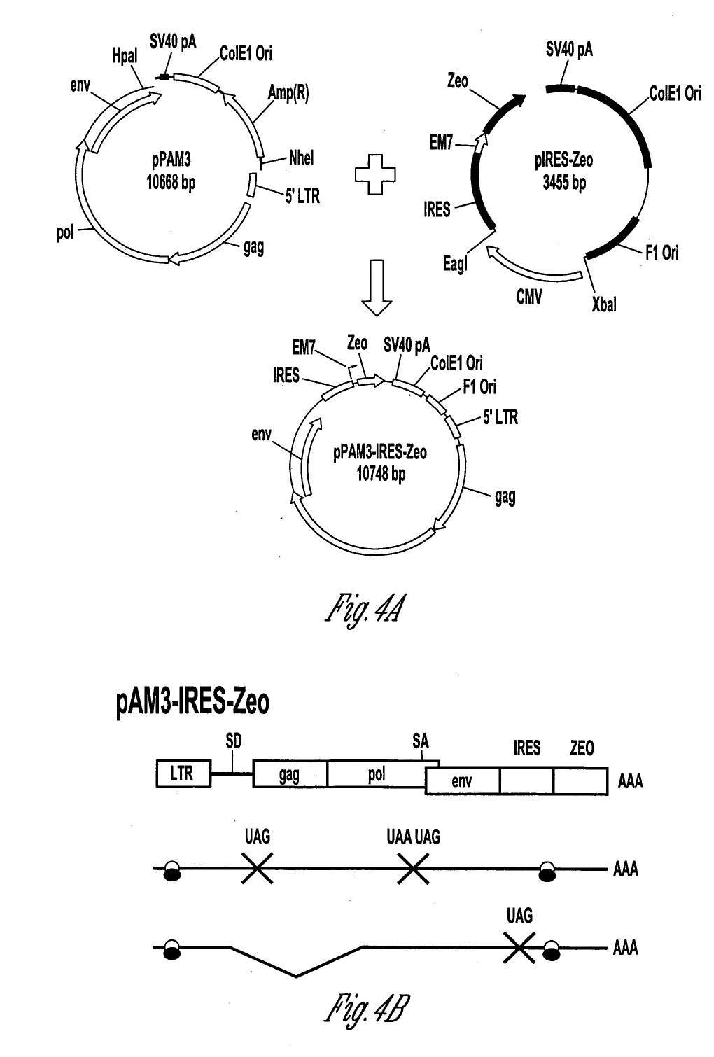

Construction of helper virus pAM3-IRES-Zeo and LEIN vector. An IRES sequence of ECMV was isolated from the LXIN retroviral vector (Clontech, Palo Alto, Calif.) by NsiI and PstI digestions and inserted into a PstI-linearized pZeoSV mammalian expression vector (Invitrogen, Carlshbad, Calif.) immediately 5′ of the EM-7 prokaryotic promoter/Zeocin™ resistance gene (Zeo) to create an IRES-Zeo expression cassette in plasmid pIRES-Zeo-SV40. SalI digestion on pIRES-Zeo-SV40 deleted the SV40 promoter and downstream polyadenylation signal to generate pIRES-Zeo. A 2.8-kb fragment, consisting of the IRES-Zeo expression cassette, SV40 polyA signal, bacterial replication origin (ColE1 Ori) and phage replication origin (F1 Ori), was excised from pIRES-Zeo by EagI digestion, Klenow fill-in (GIBCO BRL, Life Technology Co., Gaithersburg, Md.), and finally XbaI digestion. To construct pAM3-IRES-Zeo, an amphotropic helper virus pPAM3 (Miller, A. D., supra) (kindly provided by A. Dusty Miller, Fred Hutchinson Cancer Research Center, Seattle, Wash.) was digested by HpaI at 3′end of env gene and NheI at 5′end of LTR to delete the ColE1 Ori and ampicilin resistance gene (AmpR). This deleted region was replaced with the 2.8-kb IRES-Zeo fragment described above (FIG. 4). The resulting chimeric helper virus plasmid, pAM3-IRES-Zeo, allows selection with Zeocin™ in bacterial culture and mammalian cells.

The LEIN retroviral vector carrying an enhanced green fluorescent protein (EGFP) (Cormack, B. P., R. H. Valdivia, and S. Falkow. 1996. FACS-optimized mutants of the green fluorescent protein (GFP). Gene. 173:33-8; Haas, J., E. C. Park, and B. Seed. 1996. Codon usage limitation in the expression of HIV-1 envelope glycoprotein. Curr. Biol. 6:315-24; Muldoon, R. R., J. P. Levy, S. R. Kain, P. A. Kitts, and C. J. Link, Jr. 1997. Tracking and quantitation of retroviral-mediated transfer using a completely humanized, red-shifted green fluorescent protein gene. BioTechniques. 22:162-7) reporter gene was constructed by replacing the SV40 promoter-neomycin phosphotransferase gene (Neo^{r}) cassette of PLESN (27) with a 1.4-kb IRES-Neo cassette, excised from pIRES-Neo by NaeI and NsiI digestions.

Cell culture and transfection. Cell cultures were maintained in Dulbecco's modified Eagle medium (DMEM; GIBCO BRL, Life Technology Co., Gaithersburg, Md.), 10% fetal calf serum with 5% CO₂, at 37° C. The subclones of LTKOSN.2 VPC were obtained by limiting dilution of parental LTKOSN.2. VPC onto two 96-well plates (Young, W.-B., supra). Helper virus and vector gene expression, DNA methylation status and vector production in these subclones have been previously characterized (Young, W.-B., supra). To rescue LTKOSN and □LTKOSN vectors from pre-existing LTKOSN VPC subclones with methylated and silenced helper virus DNA, the subclones were transfected with pAM3-IRES-Zeo using Fugene 6™ transfection reagent (Roche Molecular Biochemicals, Indianapolis, Ind.). To study the effects of host DNA methylation on retroviral helper virus without interference from chromosomal copies pPAM3 present in LTKOSN VPC, pAM3-IRES-Zeo plasmid was transfected into NIH3T3 tk⁻ cells [American Type Culture Collection (ATCC)CRL1658] utilizing Fugene 6™ transfection reagent. A mixed population pAM3-IRES-Zeo-transfected NIH3T3 tk⁻ cells, termed AMIZ cells, was established. Prior to transfection, pAM3-IRES-Zeo plasmid was linearized by BspHI digestion and 6 to 10 μg of pAM3-IRES-Zeo was then transfected to each well in 6-well plates. Selection with Zeocin™ (350 μg/ml, Invitrogen) began 48 hr after transfection and continued for at least two weeks. Transfection of LEIN vector into the AMIZ cell pool and GP+E86 packaging cells (Markowitz, D., supra) (kindly provided by Arthur Bank, Columbia University, New York, N.Y.) was completed by DOTAP Liposomal Transfection Reagent (Roche Molecular Biohemicals) with 5 μg of LEIN plasmid for each well in 6-well plates. Selection with G418 (1 mg/ml; GIBCO) started 48 hr after transfection and continued for two weeks.

Retroviral infection, superinfection and titer assays. Supernatants collected from pAM3-IRES-Zeo-transfected LTKOSN.2 VPC subclones were diluted in 10-fold serial dilutions to transduce NIH3T3 tk cells, A375 cells (ATCC CRL1619, human melanoma) and IGROV cells [human ovarian carcinoma (Teyssier, J. R., J. Benard, D. Ferre, J. Da Silva, and L. Renaud. 1989. Drug-related chromosomal changes in chemoresistant human ovarian carcinoma cells. Cancer Genet Cytogenet. 39:35-43)], which were plated at 1×10⁵ cells/well in 6-well plate with 10 μg/ml of protamine sulfate. Twenty-four hours after transduction, cells were selected in medium containing G418 (1 mg/ml) for 10-14 days. Titers were calculated by multiplying the number of resistant colonies by the dilution factor.

To perform superinfection assays on AMIZ cells, supernatants containing LEIN vector collected from LEIN-transfected AMIZ cells were passed through a 0.4-μm syringe filter and diluted 10-fold and 100-fold before superinfection assays. Along with AMIZ cells, NIH3T3 tk⁻ and PA317 cells were transduced as Env-receptor interference negative and positive controls, respectively. Selection with G418 (1 mg/ml) on these transduced cells started 24 hr after a single exposure to LEIN vector and continued for 10-14 days. The number of G418 resistant colonies was used as the index for superinfection on PA317 and AMIZ cells. To investigate the vector production capability of AMIZ cells, a LEIN vector from ecotropic MoMLV packaging cell line, GP+E86, was transduced into AMIZ cells without further subcloning.

RNA analysis of helper virus and vector gene expression. Total cellular RNA was isolated from transfected cells and VPC by using RNA easy kit (Qiagen Inc., Valencia, Calif.) and Northern blotted from a 1% agarose-0.4M formaldehyde gel. Vector transcripts were detected by a Neo probe. Helper viral transcripts were detected by a 1.4-kb env probe, which was isolated from pPAM3 after XhoI digestion. Human glyceraldehyde 3-phosphate dehydrogenase (GAPDH) cDNA was used to demonstrate similar RNA loading and to standardize the helper virus gene expression for allow comparisons between selected and unselected cells. For analysis, the band intensities of both unspliced and spliced helper virus transcripts was divided by the intensity of GAPDH to determine relative expression levels.

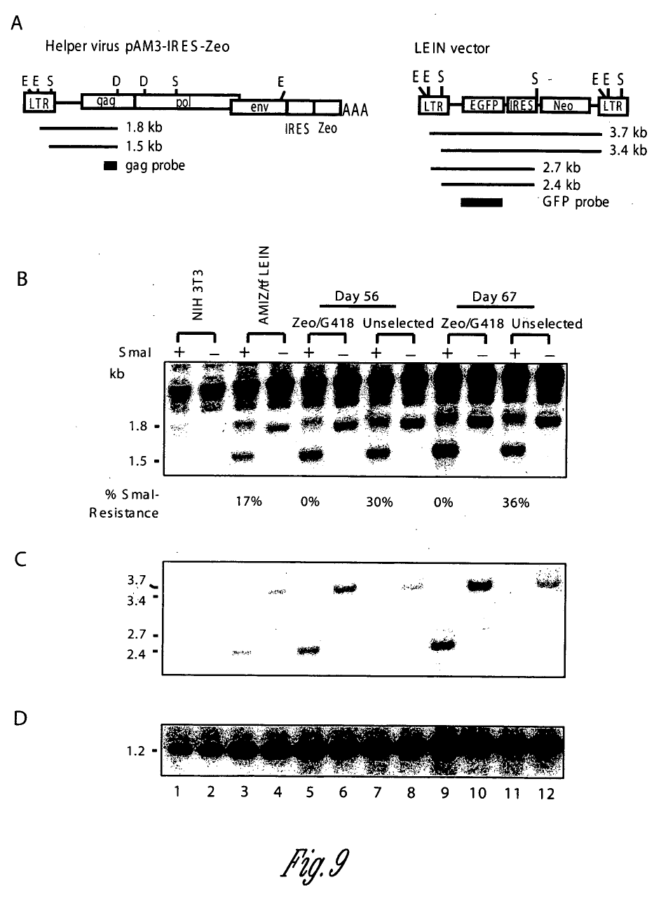

DNA Methylation analysis. In AMIZ cells transfected with LEIN vector, the methylation status of provirus and vectors was determined by evaluating the resistance to the digestion of a DNA methylation-sensitive restriction endonuclease, SmaI, in the 5′ LTR region. Genomic DNA was first digested by DraI and EcoRV to reduce DNA fragment size, precipitated with ethanol and then re-dissolved in sterile water. This DNA digest was divided into two equal portions, one of which was subjected to SmaI digestion. The Southern blot membrane was hybridized with a 428-bp fragment of gag sequence (PvuII/DraI) from pPAM3 to detect helper virus and a GFP probe to detect LEIN vector. Densitometric analyses were performed with a Hoefer Densitometer GS300 (Hoefer Scientific Instruments) to measure the relative densities of the SmaI-sensitive band compared to the DraI/EcoRV band. Due to interference from endogenous retroviral elements, the fraction of SmaI methylation in 5′ LTR was calculated as one minus the intensity ratio of the SmaI sensitive band (1.5-kb) divided by DraI/EcoRV band (1.8-kb) as depicted in FIG. 9.

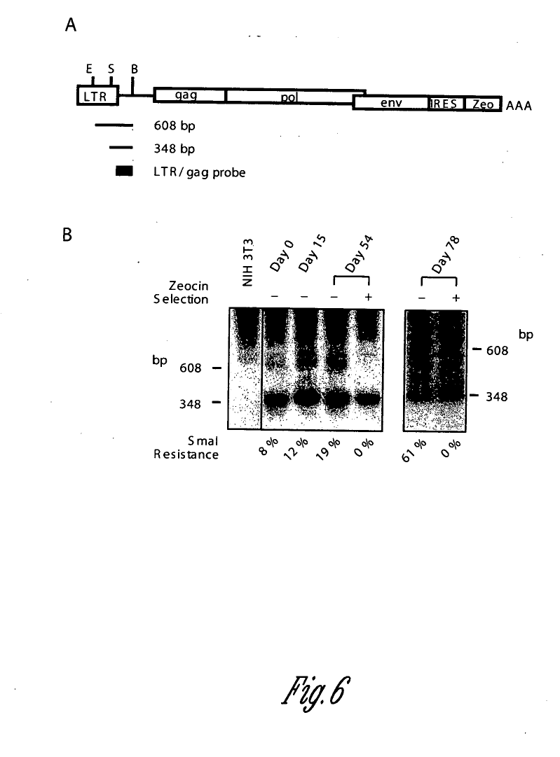

Without the interference of vector and endogenous retroviral sequences mentioned above, the DNA methylation status of the 5′LTR region of pAM3-IRES-Zeo in AMIZ cells was determined by digesting genomic DNA with EcoRV, BstEII and SmaI. If methylation occurred at the SmaI site, a 608-bp fragment would be excised instead of a 348-bp fragment when probed with a 261-bp fragment excised from pAM3-IRES-Zeo by KpnI and AflII digestions. The degree of DNA methylation was calculated as the intensity ratio of the SmaI insensitive band (608-bp) divided by the sum of the intensity of this 608-bp fragment and SmaI sensitive fragment (348-bp) as depicted in FIG. 6.

Construction of a chimeric retroviral helper virus with IRES and selection marker to allow direct selection of helper virus gene expression. We previously determined that DNA methylation occurred in 2% of the cell population per day within the 5′LTR region of helper virus to inactivate helper virus gene expression in VPC (54). To eliminate methylated helper virus 5′LTR from packaging cell population, a chimeric retroviral helper virus, pAM3-IRES-Zeo (FIG. 4), was constructed. The pAM3-IRES-Zeo construction allows Zeocin™ selection of cells with 5′LTR promoter function, since helper virus and Zeo^{r} gene expression are transcribed from the 5′LTR promoter (FIG. 4B). The selection with Zeocin™ maintains cells that also express helper virus and therefore counteract DNA methylation effects. Packaging cells based on this pAM3-IRES-Zeo helper virus should maintain high titer production. The evaluation of these pAM3-IRES-Zeo transfected cells with or without Zeocin™ selection provide a methylation profile of helper virus 5′LTR and helper virus gene expression.

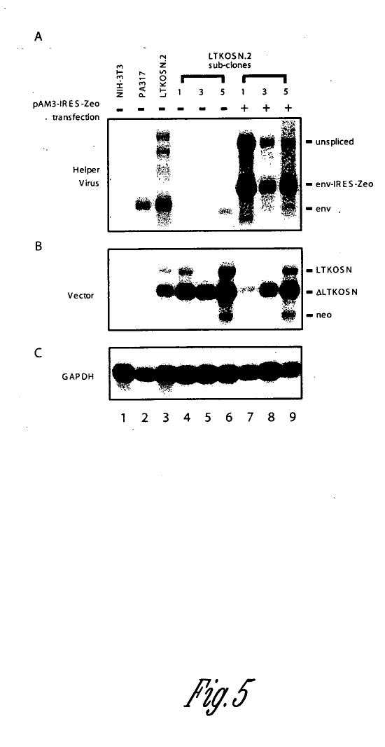

Analysis of chimeric pAM3-IRES-Zeo vector packaging ability in pre-existing LTKOSN.2 VPC subclones. To test the packaging ability of pAM3-IRES-Zeo helper virus, pAM3-IRES-Zeo was transfected into three individual subclones of LTKOSN.2 VPC. LTKOSN.2 VPC contains one LTKOSN vector and an additional ΔLTKOSN, which is derived from LTKOSN vector with an HSVtk deletion mutation (Young, W.-B., E. J. Beecham, G. L. Lindberg, and C. J. Link, Jr. 2000. Restriction mapping of retroviral vector episomal DNA. BioTechniques, in press). The pPAM3 helper virus gene expression in these three LTKOSN.2 VPC subclones #1, #3 and #5 (FIG. 5A, lanes 4-6), were inactivated by DNA methylation with impeded vector production ability (Table 1) (Young, W.-B., supra). However, LTKOSN (4.0-kb) and ΔLTKOSN (2.8-kb) vectors in these subclones were still transcribed (FIG. 5B, lanes 4-6) and no significant DNA methylation of these vectors was observed (Young, W.-B., supra). This indicated that a key limitation of vector production in LTKOSN.2 VPC subclones is the lack of helper virus gene expression. Rescue of LTKOSN and ΔLTKOSN vectors from these three subclones was performed by transfection of pAM3-IRES-Zeo followed by two weeks of Zeocin™ selection. This restored high level of vector production was shown by titer analysis on human IGROV ovarian carcinoma, human A375 melanoma and murine NIH3T3 tk⁻ target cells. The titers ranged from 4×10⁵ to 1.6×10⁷ colony forming units (cfu)/ml (Table 1). In addition, this increased packaging activity with pAM3-IRES-Zeo resulted in a reduction of retained LTKOSN and ΔLTKOSN vectors inside VPC when analyzed by Northern blot analysis (FIG. 5B, lanes 7-9).

Analysis of gene expression in pAM3-IRES-Zeo transfected LTKOSN.2 VPC subclones demonstrated significantly greater helper virus gene expression compared to pPAM3 in PA317 packaging cells and parental LTKOSN.2 VPC (FIG. 5A). In addition to env transcripts, only one population of unspliced helper virus (gag-pol-env-IRES-Zeo) was detected in pAM3-IRES-Zeo transfected subclones, which indicates that the integration of pAM3-IRES-Zeo should be intact in transfected cells after selection. In contrast, co-transfection of pPAM3 without direct selection for pPAM3 gene expression but other selection marker in trans could result in randomly interrupted pPAM3 for integration. This was shown by two additional transcripts of lower molecular weight detected in PA317 and LTKOSN.2 VPC (FIG. 5, lanes 2 and 3) (Young, W.-B., supra). These results demonstrate that enhanced and sustained helper virus gene expression can be obtained in polyclonal packaging cells when pAM3-IRES-Zeo is used to allow Zeocin™ selection without the need to perform time-consuming cell subcloning. This implies a potential use of pAM3-IRES-Zeo to establish new packaging cells from other cells such as human primary cells.

Cells transfected with pAM3-IRES-Zeo provide a model to study DNA methylation of retroviral sequences. DNA methylation in mammalian cells is site-dependent within the genome (Hoeben, R. C., A. A. Migchielsen, R. C. van der Jagt, H. van Ormondt, and A. J. van der Eb. 1991. Inactivation of the Moloney murine leukemia virus long terminal repeat in murine fibroblast cell lines is associated with methylation and dependent on its chromosomal position. J. Virol. 65:904-12). Therefore, a mixed population of pAM3-IRES-Zeo transfected cells would be required to study DNA methylation of helper virus 5′LTR to minimize the effects of positional interference. To establish a pooled population of packaging cells without chromosomal pPAM3 effects, pAM3-IRES-Zeo was transfected into NIH3T3 tk⁻ cells followed by selection with Zeocin™ and without further subcloning. This pool of newly established packaging cells was named AMIZ packaging cells (pAM-IRES-Zeo). To allow DNA methylation to occur, AMIZ cells were released from Zeocin™ selection for one month and then placed in continuous culture with or without Zeocin™ selection for 78 days (10 passages). DNA methylation and gene expression of pAM3-IRES-Zeo were examined at 15, 54, and 78 days after being released from selection. Over the first 54 days of cell culture period, DNA methylation of 5′LTR was increased from 8% to 19% and by Day 78 reached 61% (FIG. 6). The DNA methylation rate of helper virus 5′LTR averaged 0.7% of population per day during 78 day period. AMIZ cells with continued Zeocin™ selection did not exhibit any detectable DNA methylation (FIG. 6). This drug selection effectively eliminated methylated pAM3-IRES-Zeo from the pooled AMIZ population.

Retroviral superinfection is blocked by enhanced helper virus gene expression. The effect of Zeocin™ selection on AMIZ cells was analyzed by gene expression of pAM3-IRES-Zeo in AMIZ cells. Gene expression of pAM3-IRES-Zeo in AMIZ cells with constant Zeocin™ selection showed a 2-fold increase compared to AMIZ cells without selection on Day 15 and at least 4-fold increase on Days 54 and 78 (FIG. 7). In contrast, pAM3-IRES-Zeo gene expression in AMIZ cells without Zeocin™ selection declined over time (FIG. 7, lanes 3, 5 and 7). Continuous Zeocin™ selection may have selected integration sites that are highly transcriptionally active and have less DNA methylation activity (Cedar, H. 1988. DNA methylation and gene activity. Cell. 53:3-4; Keshet, I., supra).

We directly determined whether decreased pAM3-IRES-Zeo gene expression reduced Env-receptor interference and increased vector superinfection. The susceptibility to superinfection was measured by exposing AMIZ cells from the above experiment to amphotropic LEIN vector supernatants followed by G418 selection. G418 resistant colony number obtained from AMIZ cells with continued Zeocin™ selection was reduced from 2.3×10¹ on Day 15 to no superinfection observed on Days 54 and 78 (Table 2). In contrast, G418 resistant colonies obtained from AMIZ cells without Zeocin™ selection ranged from 1.2×10³ to 5.6×10³. These results demonstrate that increased gene expression of helper virus correlates with reduced susceptibility to superinfection.

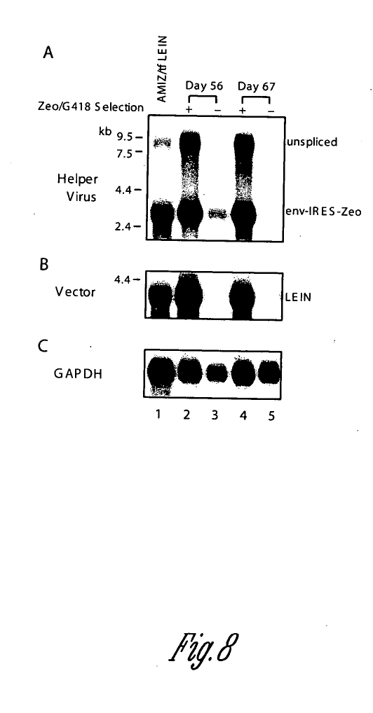

High level of vector production was maintained by Zeocin™ selection. Vector production was analyzed in this AMIZ cell pool by transfecting LEIN vector into AMIZ cells followed by G418 selection to establish a VPC for titer assay. Zeocin™ selection was temporally withdrawn from AMIZ cell culture during the first three weeks of G418 selection after transfection with LEIN vector. Titer obtained from this newly established uncloned population of AMIZ cells was 3.5×10⁶ cfu/ml, which is 100-fold higher than the titer observed from a mixed population of PA317 transfected with LEIN vector (4×10⁴ cfu/ml). In addition, AMIZ cells were transduced with LEIN vector collected from LEIN-transfected GP+E86 cells and an improved titer of 9×10⁶ cfu/ml was obtained from a mixed cell population. To investigate whether selection with both Zeocin™ and G418 would adversely affect vector production, LEIN transfected AMIZ cells were evaluated 56 (8 passages) and 67 days (10 passages) after transfection. Titers obtained from AMIZ cells transfected with LEIN (3.5×10⁶ cfu/ml on Day 0) and placed under continuous selection with Zeocin™ and G418, were 2×10⁶ cfu/ml (Day 56) and 1.5×10⁷ cfu/ml (Day 67). In contrast, titers obtained from the same AMIZ cells transfected with LEIN but not subjected to G418 and Zeocin™ selection only showed 2×10⁴ and 4×10⁴ cfu/ml on Day 56 and Day 67, respectively. The reduced titer correlated with a significant decrease of both helper virus and vector gene expression when time points with and without selection were compared (FIG. 8). No significant increase of titer or helper virus gene expression was observed when the 17% DNA methylation present on Day 0 was further reduced to 0% DNA methylation by Day 56 after selection. This suggests a threshold effect as we previously observed in cloned VPC (Young, W.-B., supra). Substantial decreases of vector production, helper virus gene expression and Env-receptor interference was only observed once at least 60% methylation occurred of the helper virus 5′LTR.

DNA methylation status of 5′LTRs of helper virus and vector were significantly increased in AMIZ cells transfected with LEIN vector and cultured without either G418 or Zeocin™ selection (FIG. 9). This increased methylation corresponded to above decreased vector titer and significantly reduced gene expressions of helper virus and vector (FIG. 8). The DNA methylation of helper virus 5′LTR increased from 17% (Day 0) to 30% and 36% by Days 56 and 67, respectively. The average DNA methylation rate of helper virus 5′LTR in AMIZ cells transfected with LEIN was estimated as low as 0.3% of the cell population per day during 67 days of continuous cell culture. In contrast, DNA methylation was not detected in AMIZ cells transfected with LEIN vector and placed under continuous G418 and Zeocin™ selection. No detectable DNA methylation occurred in the LEIN vector on Day 0 (FIG. 9C, lanes 3 and 4) while the 5′LTR helper virus showed 17% DNA methylation (FIG. 9B, lanes 3 and 4). This may be secondary to the timing of G418 and Zeocin™ selection. AMIZ cells transfected with LEIN vector were placed under G418 for three weeks to select for LEIN-positive population and Zeocin™ selection was not applied until Day 0 in the experiment.

The experimental model described has demonstrated an approach using a retroviral helper virus combining a picornavirus IRES sequence and a selection marker gene that allows efficient elimination of methylated helper virus from packaging cell populations. This strategy of using drug selection maintained high levels of helper virus gene expression and high titer vector production (1.5×10⁷ cfu/ml) from non-subcloned population of VPC. The presence of greater Env-receptor interference blocks vector superinfection and may reduce other potential problems with retroviral vectors including RCR formation and multiple copies of vectors. A new packaging cell pool, AMIZ cells, established by transfection of pAM3-IRES-Zeo chimeric helper virus into NIH3T3 tk⁻ cells without any subcloning procedure, has proved a useful system to study the effect of host DNA methylation on retroviral sequences.

The selection of transfected cells (AMIZ cells) with Zeocin™ to maintain pAM3-IRES-Zeo gene expression eliminated DNA methylation from AMIZ cells and may also select cells with pAM3-IRES-Zeo helper virus integrated in optimal and active chromosomal regions. Ratios of pAM3-IRES-Zeo gene expression in selected AMIZ cells compared to non-selected AMIZ cells were about 2:1 on Day 15 and at least 4:1 on Days 54 and 78 (FIG. 7), while helper virus showed only 12%, 19% and 61% of DNA methylation, respectively (FIG. 6). Similar results were also observed in AMIZ cells transfected with LEIN vector. Cells under continuous selection showed no detectable DNA methylation of the 5′LTR, but 30% (Day 56) and 36% (Day 67) of DNA methylation was detected in cells without selection (FIG. 9). LEIN-transfected AMIZ cells under continuous selection had vector titer of 1.5×10⁷ cfu/ml on Day 67, compared to 4×10⁴ cfu/ml on Day 67, in cells without selection. This 1000 fold difference in titer production probably reflects the fact that structural proteins of viruses function as multimers (Hunter, E., supra). The formation of multimers occurs in a sigmoid dose-response fashion, rather than a linear dose-response to protein concentration that correlates more directly with helper virus gene expression and DNA methylation. The effect of host DNA methylation on helper virus 5′LTR is therefore amplified by transcription, viral assembling and then vector production.

To maintain efficient Env-receptor interference and active viral production, a threshold level of helper virus gene expression is required. In retrovirus infection, this threshold level of gene expression is established by the accumulation of sufficient copy number of virus through superinfection until efficient Env-receptor interference is achieved and maintained (Odawara, T., supra). In our study, the threshold level of helper virus gene expression was achieved by Zeocin™ selection rather than increasing the copy number of helper virus. Superinfection was observed when selection pressure was released and helper virus gene expression declined. These results support a conclusion that continuous selection of helper virus in VPC might enhance Env-receptor interference and reduce the possibility of RCR formation.

For continuous virus production, retroviral gene expression has to be regulated at sufficient level without interfering with host cell growth and differentiation. Increased levels of viral RNAs and proteins in infected cells can cause cytopathic effects, usually at the cost of cell death, by interrupting the production or translation of host mRNA (Somasundaran, M., and H. L. Robinson. 1988. Unexpectedly high levels of HIV-1 RNA and protein synthesis in a cytocidal infection. Science. 242:1554-7). Although we observed that AMIZ cells under continuous selection did proliferate more slowly than AMIZ cells without selection, AMIZ cells under continuous G418 and Zeocin™ selection for high gene expression for 67 days (FIG. 8) still proliferated (data not shown). We did not attempt to select for pPAM3 gene expression by drug selection against the HSVtk selection marker plasmid co-transfected into PA317 cells. This approach is unlikely to be successful since the selection marker plasmid is separate from pPAM3. An alternative approach to reverse methylation is treatment with 5′-aza-cytidine (5-aza-C) to reverse DNA methylation (Juttermann, R., E. Li, and R. Jaenisch. 1994. Toxicity of 5-aza-2′-deoxycytidine to mammalian cells is mediated primarily by covalent trapping of DNA methyltransferase rather than DNA demethylation. Proc. Natl. Acad. Sci. USA. 91:11797-11801; Lengauer, C., K. W. Kinzler, and B. Vogelstein. 1997. DNA methylation and genetic instability in colorectal cancer cells. Proc. Natl. Acad. Sci. USA. 94:2545-50). In previous experiments, we found that only a minor portion of pPAM3 helper virus expression could be restored by 5-aza-C (Young, W.-B., supra). Treatment with 5-aza-C does not specifically reverse helper virus DNA methylation, but also inhibits cellular DNA methyltransferase and causes cytotoxicity to treated cells (Juttermann, R., supra). The data from this study suggest that a combination of helper virus and IRES sequences with selectable markers is a viable option to eliminate host DNA methylation of helper virus from VPC.

One benefit of this chimeric helper virus to gene therapy would be to allow packaging cells to be established from primary cell culture without subcloning. This might be useful for transplanting VPC into patients (Ram, Z., supra) without the immune elimination of murine VPC and virions (Takeuchi, Y., supra; Takeuchi, Y., supra). Several studies have aimed to establish a retroviral packaging cell line by using either adenovirus (Caplen, N. J., J. N. Higginbotham, J. R. Scheel, N. Vahanian, Y. Yoshida, and H. Hamada. 1999. Adeno-retroviral chimeric viruses as in vivo transducing agents. Gene Ther. 6:454-459; Feng, M., W. H. Jackson, Jr., C. K. Goldman, C. Rancourt, M. Wang, S. K. Dusing, G. Siegal, and D. T. Curiel. 1997. Stable in vivo gene transduction via a novel adenoviral/retroviral chimeric vector. Nat. Biotechnol. 15:866-70; Lin, X. 1998. Construction of new retroviral producer cells from adenoviral and retroviral vectors. Gene Ther. 5:1251-8) or herpes simplex virus (Savard, N., F. L. Cosset, and A. L. Epstein. 1997. Defective herpes simplex virus type 1 vectors harboring gag, pol, and env genes can be used to rescue defective retrovirus vectors. J. Virol. 71:4111-7), to import retroviral helper virus genome into target cells in vivo or ex vivo. In this study, chimeric retroviral helper virus, pAM3-IRES-Zeo was used to generate a pooled population of pAM3-IRES-Zeo-transfected cells, AMIZ cells. AMIZ cells transfected with a retroviral vector maintained titers between 3.5×10⁶ to 1.5×107 cfu/ml. These titers are comparable to reported titers from individually cloned VPC, which generally ranged from 10⁴ to 10⁷ cfu/ml (Miller, A. D. 1990. Retrovirus packaging cells. Hum. Gene Ther. 1:5-14). Transfection of the pAM3-IRES-Zeo into cells followed by selection for positive populations can take only two weeks or less, depending on transfection efficiency. Since some primary cell cultures are too sensitive to allow effective antibiotic selection, replacing Zeocin™ selection marker with a cellular surface marker or GFP gene might be required to overcome obstacles to making VPC from primary cell lines.

### EXAMPLE 3

### Results of Cloning of Stably Transfected Retroviral VPC (A375.AMIZ-1/LNL and A375.AMIZ-2/LNL) by Limiting Dilutions

History of the Project

1. A375.NV human malanoma cell line was transfected with packaging construct designed by Won Bin Young (pPAM-IRES-Zeo) and selected with 350 μg/ml Zeo. This resulted in 2 clones (Zeo resistant) A375.AMIZ-1 and A375.AMIZ-2. Starting from first passages A375.AMIZ-2 had doubling time approximately twice shorter when equal number of cells for each line was seeded (2×10⁶/T80 flask) and counted after 72 hours.

2. A375.AMIZ-1 and A375.AMIZ-2 packaging cell lines were each transfected with LNL construct (MSEV LTR construct by Won Bin and Bob Unfer) in 6 well plates. Each transfection well was carried individually through G418 selection (1 mg G418/ml and 48 hour supsernatants from each stably transfected subculture (at least 14 days of G418 selection) were titered on Igrov.NV cells.

3. Two best mixed populations with highest titers, one of each A375.AMIZ-1/LNL and A375.AMIZ-2/LNL, were chosen for cloning by limiting dilutions. The titers of best A375.AMIZ-1/LNL and A375.AMIZ-2/LNL were similar and approximately 1-2×10⁵/ml.

4. After plates for cloning were seeded we found out that Igrov.NV on which titration was performed was contaminated with mycoplasma. This finding and results of recent titrations in which the same positive control supe used previously for phase II LTROSN/1 gives titers at least two times higher then on mycoplasma positive IGROVS, allows make an assumption that real titers of stably transfected A375.AMIZ/LNL cultures were higher than 1-2×10⁵.

5. On Feb. 8, 2000 cloning of A375.AMIZ-1/LNL and A375.AMIZ-2/LNL subcultures with highest titers was performed by limiting dilution:
cells were plated into 96-well plates
No G418/or Zeo in D10 at concentration 5 cells/ml 200 μl/well (equal 1 cell/well)

$$- A375 \cdot AMIZ - 1 / LNL : \frac{78}{4} \times 10 \times {10}^{4} = 1.95 \times {10}^{6} cells / ml$$

Dilutions:
1 ml 1.95×10⁶+0.95 ml D10=1×10⁶/cells/ml
0.2 ml 1×10⁶/ml+1.8 ml D10=2 ml 1×10⁵/ml
0.2 ml 1×10⁵/ml+1.8 ml D10=2 ml 1×10⁴/ml
0.2 ml 1×10⁴/ml+1.8 ml D10=2 ml 1×10³/ml
1 ml of 1×10³ cells/ml+199 ml D10=200 ml at 5 cells/ml 20 ml/plate→approximately 10 plates

$$- A375 \cdot AMIZ - 2 / LNL : \frac{60}{4} \times 10 \times 104 = 1.5 \times 106 cells / ml$$

Dilutions and plating: see above for A375.AMIZ-1/LNL

6. Clones were registered first time on Feb. 15, 2000. Wells which were claimed to be single clones on Feb. 15, 2000 were rechecked and reconfirmed on Feb. 18, 2000.

7. Majority of positive wells were trypsinized at least once to ensure good spread of cells in a well required for development of a good monolayer for collection of a supe for titer screen.

8. Preliminary titer screening was performed as follows:
D10 in 90-95% confluent wells was completely replaced with 250 μl of fresh DIO (NO G418 or Zeo) for 48 hours.

9. After 48 hours supe was collected into 2 separate aliquotes: 40 μl—for titers on Igrov, and 210 μl (all what is left)—for slot blotting aliquotes were collected into small eppendorfs. Cells from the corresponding well were trypsinized, harvested into 2 ml, pelleted, resuspenced in 400 μl of 90% FBS+1-% DMSO and frozen in one aliquote in small eppendorf (the same type as for supes). Empty boxes from 1000 μl tips were used for storage of eppendorfs with supes and cells at −70°.

12. The slowest growing wells which could be confluent after Mar. 1, 2000 were not persued.

TABLE 3
General information on results of cloning by limiting dilutions

| | | Wells with Cells | | | | | | | | | |
|---|---|---|---|---|---|---|---|---|---|---|---|
| | | | % of | 1 clone well | | 2 clone well | | 3 clone wells | | | |
| | Total | | Total | | % of | | % of | | % of | Total wells | |
| | # of | | # of | | positive | | positive | | positive | harvesting | |
| Cloner | wells | # | wells | # | wells | # | wells | # | wells | (% position) | |
| #1 | 384 | 233 | 61% | 118 | 51% | 72 | 31% | 43 | 18% | 83 | (36%) |
| #2 | 156 | 96 | 62% | 36 | 37% | 32 | 33% | 28 | 30% | 34 | (35%) |
| #3 | 579 | 331 | 58% | 199 | 60% | 86 | 26% | 46 | 14% | 91 | (28%) |
| #4 | 384 | 262 | 68% | 158 | 60% | 81 | 31% | 23 | 9% | 103 | (39%) |
| #5 | 384 | 231 | 60% | 130 | 56% | 79 | 34% | 22 | 10% | 138 | (60%) |
| TOTAL | 1,884 | 1,153 | 61% | 641 | 55% | 350 | 30% | 165 | 14% | 448 | (39%) |

TABLE 4
Results of slot-blot screening of harvested 48 hour supes trom positive wells

| | | | intensity score by slot-blot | | | | | | | | | | | | | | | | | | | |
|---|---|---|---|---|---|---|---|---|---|---|---|---|---|---|---|---|---|---|---|---|---|---|
| Mixed | | Total | 0 | | 1 | | 2 | | 3 | | 4 | | 5 | | 6 | | 7 | | 8 | | 9 | |
| popu- | | Ana- | | % | | % | | % | | % | | % | | % | | % | | % | | % | | % |
| lation | | lyzed | | of | | of | | of | | of | | of | | of | | of | | of | | of | | of |
| origin | Cloner | (TA) | # | TA | # | TA | # | TA | # | TA | # | TA | # | TA | # | TA | # | TA | # | TA | # | TA |
| A375. | #1 | 83 | 2 | 2% | 27 | 33% | 7 | 8% | 9 | 11% | 3 | 4% | 7 | 8% | 9 | 11% | 16 | 19% | 3 | 4% | 0 | 0% |
| AMIZ- | #3 | 91 | 3 | 3% | 37 | 41% | 9 | 10% | 6 | 6% | 4 | 4% | 5 | 5% | 10 | 11% | 13 | 14% | 2 | 2% | 2 | 2% |
| 2/LNL | TOTAL | 174 | 5 | 3% | 64 | 37% | 16 | 9% | 15 | 9% | 7 | 4% | 12 | 7% | 19 | 11% | 29 | 17% | 5 | 3% | 2 | 1% |
| A375. | #2 | 34 | 0 | 0% | 6 | 18% | 8 | 23% | 10 | 29% | 6 | 18% | 2 | 6% | 1 | 3% | 1 | 3% | 0 | 0% | 0 | 0% |
| AMIZ- | #4 | 103 | 1 | 1% | 3 | 3% | 22 | 21% | 41 | 40% | 11 | 11% | 12 | 12% | 10 | 10% | 2 | 2% | 1 | 1% | 0 | 0% |
| 1/LNL | #5 | 138 | 6 | 4% | 29 | 21% | 38 | 27% | 37 | 27% | 18 | 13% | 8 | 6% | 0 | 0% | 2 | 1% | 0 | 0% | 0 | 0% |
| | TOTAL | 275 | 7 | 2% | 38 | 14% | 68 | 24% | 88 | 32% | 35 | 13% | 22 | 8% | 11 | 4% | 5 | 2% | 1 | 0.3% | 0 | 0% |

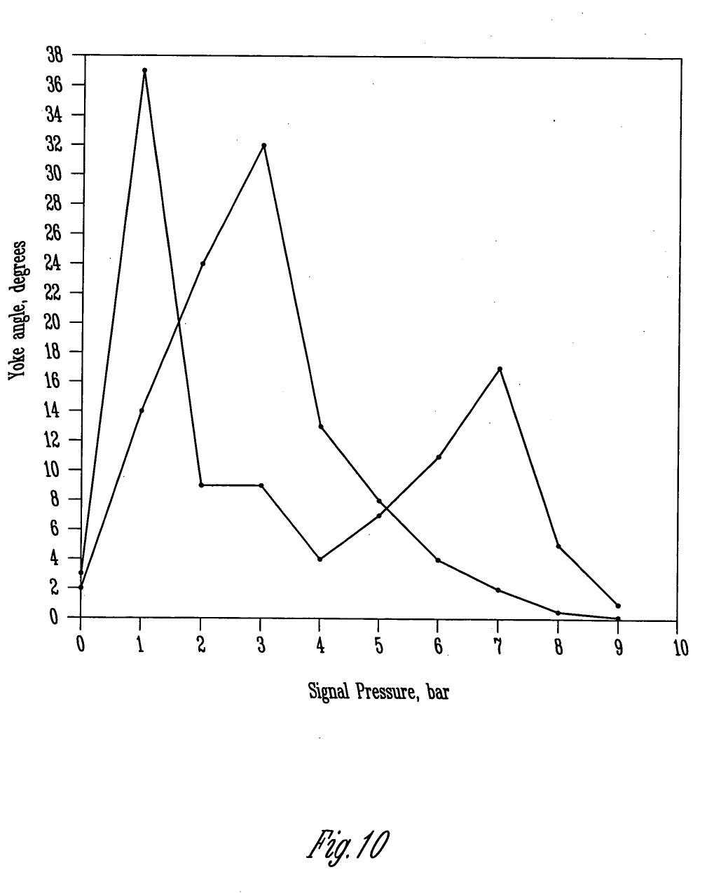

Distribuion of VPC microcultures are function of slot-blot intensity score for VPC derived from A375.AMIZ-1 and A375.AMIZ-2 packaging cell lines is depictured on FIG. 10. A375.AMIZ-2 has doubling time approx. twice shorter than A375.AMIZ-1. Medium score (from 0 to 9) is 4.5.

| | | |
|---|---|---|
| # of cultures scored | A375.AMIZ-2/LNL | A375.AMIZ-1/LNL |

| | | | | |
|---|---|---|---|---|
| below average: | 107 | (61.5%) | 236 | (86%) |
| above average: | 67 | (38.5%) | 39 | (14%) |

III. Selection of Best Cultures for the Future Clinical Grade VPC Clone

1. Based on slot-blot score results all cultures with score 7 and up are expanded into T80 and supes for titers are collected as follows:
2×10⁶/T80 seeded→90-95% confluency→→10 ml D10 for 48 hours→supe collection centrifugation (10 min, 3,000 rpm)→cell harvesting→freezing: 8 aliquots of cells (seed bank)→cells are split into 2×T80 of supe and continued for cell harvest for DNA (Southern and HIV etc. PCR).

2. Supes are titerd on Igrovs in triplicates:
according to preliminary titer data on Barbara's single clone cultures (she expanded them from 96 wells into T25 and collected supes at 48 hours) there are some clones with titers higher than 1×10⁶. Because of that supes will be titered in triplicates (10⁻⁴, 10⁻⁵, 10⁻⁶ dilution)

1. A method for increasing viral vector titer in a vector packaging cell comprising:
contacting a vector packaging cell with a viral vector, said viral vector comprising a viral packaging signal sequence and a nucleotide sequence, the presence of which is desired in a host cell, said packaging cell capable of expressing structural viral components so that said viral vector may be assembled to form an infectious viral particle; and
inhibiting the presence of 5′ methylated helper virus in said cell.

2. The method of claim 1 wherein said step of inhibiting methylation comprises:
positively selecting helper virus which is functional.

3. The method of claim 2 wherein said selection is by antibiotic resistance.

4. The method of claim 3 wherein said antibiotic resistance selection is accomplished via ligation of an internal ribosome entry site with a selection marker so that drug selection ensures promoter function in said helper virus.

5. The method of claim 1 wherein said viral titer achieves levels of 1.5×10⁷ cfu/ml in the presence of antibiotic resistant selection.

6. The method of claim 1 wherein said helper virus comprises at least one viral production gene operably linked to viral promoter sequence which is capable of being methylated.

7. The method of claim 6 wherein said viral promoter comprises a long term repeat.

8. The method of claim 7 wherein said viral promoter sequence is the LTR of retrovirus.

9. The method of claim 1 wherein said step of decreasing inactive helper virus comprises the step of:
inhibiting methylation of helper virus.

10. The method of claim 9 wherein said inhibiting of methylation is accomplished by: a step selected from the group consisting of:
treating of vector producer cells with 5-AZA-C.

11. The method of claim 9 wherein said inhibiting of methylation is accomplished by: a step selected from the group consisting of:
insertion of a demethylation fragment of murine thy-1 in front of the 5′ long terminal repeat.

12. The method of claim 9 wherein said inhibiting of methylation is accomplished by: a step selected from the group consisting of:
immune response selection.

13. The method of claim 9 wherein said inhibiting of methylation is accomplished by: a step selected from the group consisting of:
design of synthetic viral promoters to omit methylation sites.

14. The method of claim 9 wherein said inhibiting of methylation is accomplished by: a step selected from the group consisting of:
introducing a drug which inhibit methylation

15. The method of claim 9 wherein said inhibiting of methylation is accomplished by: a step selected from the group consisting of:
antisense inhibition of the human methylate gene.

16. A helper virus nucleotide sequence comprising:
a packaging deficient nucleotide sequence which encodes one or more structural viral components necessary for assembling a viral capsid, a viral promoter sequence capable of becoming methylated and a marker selection gene placed so that active helper virus may be positively selected.

17. The nucleotide sequence of claim 16 wherein said viral promoter sequence comprises:
a long terminal repeat promoter sequence which has been modified to inhibit methylation.

18. The helper virus nucleotide sequence of claim 16 wherein said sequence includes the methylation fragment of murine thy-1 in front of the 5′ long terminal repeat site.

19. The helper virus nucleotide sequence of claim 16 wherein said sequence includes an internal ribosome entry site with a selection marker downstream of a viral component encoding sequence so that selection ensures promoter function.

20. The helper virus nucleotide sequence of claim 16 wherein said internal ribosome entry site is a picornavirus internal ribosome entry site.

21. The helper virus nucleotide sequence of claim 16 wherein said marker selection gene is an antibiotic resistance marker.

22. The helper virus nucleotide sequence of claim 16 wherein said helper virus is pAM3-IRES-Zeo.

23. A vector packaging cell, said cell comprising a helper virus nucleotide sequence according to claim 16.

24. A vector producer cell comprising a helper virus nucleotide sequence according to claim 16 and a viral vector, said producer cell capable of assembling virions particles.

25. An infectious viral particle produced by the method of claim 1.

26. A method for increasing viral titer produced by a vector packaging cell upon transfection with a viral vector comprising:
decreasing the amount of inactive helper virus present in said vector packaging cell by providing for the elimination of or prevention of methylated helper virus.

27. The method of claim 26 wherein said step of decreasing inactive helper virus comprises the step of:
inhibiting methylation of helper virus.

28. The method of claim 26 wherein said step of decreasing inactive helper virus comprises the step of:
removing from a population of vector packaging cells, helper virus with 5′ long terminal repeat methylation.

29. The method of claim 28 further comprising the step of:
removing cells with inactivated virus by positive selection.

30. The method of claim 29 wherein said removal step comprises:
introducing an antibiotic to to said cells so that cells with inactive helper virus are killed.

31. The method of claim 30 wherein said removal is accomplished by a helper virus with a picarnovirus internal ribosomal entry site sequence followed by an antibiotic resistance marker at the 3′ end of the env sequence of said helper virus.

32. The method of claim 31 wherein said antibiotic resistance selection marker is Zeocin.

33. The method of claim 27 wherein said inhibiting of methylation is accomplished by a step selected from the group consisting of:
treating of vector producer cells with 5-AZA-C.

34. The method of claim 27 wherein said inhibiting of methylation is accomplished by a step selected from the group consisting of:
insertion of a demethylation fragment of murine thy-1 in front of the 5′ long terminal repeat.

35. The method of claim 27 wherein said inhibiting of methylation is accomplished by a step selected from the group consisting of:
ligation of an internation ribosome entry site with a selection marker so that drug selection would ensure promoter function.

36. The method of claim 27 wherein said inhibiting of methylation is accomplished by a step selected from the group consisting of:
immune response selection.

37. The method of claim 27 wherein said inhibiting of methylation is accomplished by a step selected from the group consisting of:
design of synthetic viral promoters to omit methylation sites.

38. The method of claim 27 wherein said inhibiting of methylation is accomplished by a step selected from the group consisting of:
screening for and identification of other drugs which inhibit methylation.

39. The method of claim 27 wherein said inhibiting of methylation is accomplished by a step selected from the group consisting of:
an antisense inhibition of the human methylate gene.
